SEQUENCE LISTING (4 sequences)

SEQ ID NO: 1 - protein, 9 aa, Homo sapiens
LLLTVLTVV

SEQ ID NO: 2 - protein, 9 aa, Homo sapiens
IMDQVPFSV

SEQ ID NO: 3 - protein, 9 aa, Homo sapiens
KTWGQYWQV

SEQ ID NO: 4 - protein, 9 aa, Homo sapiens
AAGIGILTV

(19) United States
(12) Patent Application Publication
(10) Pub. No.: US 2008/0213237 A1
(43) Pub. Date: Sep. 4, 2008

(54) Antigen Specific Lymphocytes, Compositions Thereof, and Methods for Isolation and Preparation Thereof

(75) Inventors: Shoshana Frankenburg, Jerusalem (IL); Jacob Pitcovski, Korazim (IL); Tamar Peretz, Jerusalem (IL); Michal Lotem, Reut (IL); Arthur Machlenkin, Kiryat Eqron (IL); Ronny Uzana, Rehovot (IL)

Correspondence Address: BROWDY AND NEIMARK, P.L.L.C.;624 NINTH STREET, NW, SUITE 300, WASHINGTON, DC, 20001-5303, US

(73) Assignees: Hadasit Medical Research Services and Development Ltd., Jerusalem (IL); Gavish Galilee Bio Applications Ltd., Kiryat Shmona (IL)

(21) Appl. No.: 11/908,279

(22) PCT Filed: Mar. 9, 2006
(86) PCT No.: PCT/IL06/00313

§ 371 Date: Apr. 14, 2008

(30) Foreign Application Priority Data
Mar. 10, 2005 (IL) 167388

(51) Int. Cl. A61K 35/12; C12Q 1/02; G01N 33/53; A61P 43/00; A01K 67/027; C12N 5/06

(52) U.S. Cl. 424/93.71; 435/29; 435/7.24; 800/3; 435/372.3

(57) ABSTRACT

The invention relates to a method for the isolation of T cell lymphocyte, preferably, CD8⁺ cytotoxic T lymphocyte, which is capable of specifically recognizing an antigen related to a pathologic disorder. The method of the invention is base on ability of the CTL's to capture membrane from labeled target cells. The invention further provides compositions comprising said specific lymphocytes and methods for the treatment of said pathologic disorder using the specific lymphocytes isolated and prepared by the method of the invention.

### FIELD OF THE INVENTION

The invention relates to a method for the isolation of T cell lymphocytes specific for an antigen. More particularly, the invention relates to a method for the isolation of CD8⁺ cytotoxic T lymphocytes specific for antigens associated with a pathologic disorder, preferably, an immune related disorder. The invention further provides compositions comprising said specific lymphocytes and methods for the treatment of said pathologic disorder using the specific lymphocytes isolated and prepared by the method of the invention.

### BACKGROUND OF THE INVENTION

A major part of research in cancer vaccines is concentrated on detection of antigens that are presented preferentially or solely on tumor cells. Presentation of a specific peptide to the relevant cytotoxic T lymphocyte (CTL) in the context of MHC class I molecules on the antigen presenting cells (APC) induces the propagation of specific CTL, and enhances their potential to kill tumor cells that present such particular peptide. Identification of specific peptides enables the development of vaccines that enhance immune cells (e.g. CD8+ lymphocytes) mediated killing of tumor cells presenting those peptides.

Peptides may be presented on MHC class I by three routes. In vivo, most of the peptides that are found on MHC class I are derived from proteins that were produced in the cell and degraded in the proteasome. Lately it was found that APC can present peptides of proteins that were taken up by the cells. In vitro, in addition to those two ways, MHC class I may be loaded externally with a synthetic peptide and elicit CTL.

The discovery of tumor associated antigens (TAA) was a defining point in tumor immunology, as it represented the first step towards rational design of cancer immunotherapy. It is now known that TAA recognized by cytotoxic T lymphocytes (CTL) in the context of MHC class I molecules represent a critical component of the immune response against the tumor. Most techniques for identification of tumor antigens involve prolonged in vitro culture of T cells in the presence of tumor cells. It has been suggested that these methods, in addition to being long and cumbersome, may skew toward the identification of inferior tumor antigens [Gilboa E. Immunity 11, 263-70 (1999)]. In addition, high avidity cytotoxic T lymphocytes corresponding to immunodominant epitopes are less likely to survive this process [Alexander-Miller M A, et al., J. Exp. Med. 184, 485-92 (1996)].

More particularly, it is surprising that the extensive database of antigen-derived epitopes accumulated so far, is not backed up with practical technologies for quantitative identification and isolation of epitope-reactive T cells. The common starting point employed by researchers who aimed at cloning antigen-specific T cells has been repeated stimulations of bulk mononuclear cells. Thus, there is a preferential selection and growth of antigen-reactive T cells, which may be as scarce as one in 100,000 peripheral blood T cells [Chen W. et al., J. Exp. Med. 193, 1319-26 (2001)], as indicated above. In addition to being long and cumbersome, T cell culture assays have several pitfalls. High avidity cytotoxic T lymphocytes corresponding to immunodominant epitopes are less likely to survive repeated antigenic stimulations [Alexander-Miller (1996) ibid.], T cells reactive to inferior antigenic epitopes may dominate the cultures [Gilboa (1999) ibid]. Furthermore, Ag-reactive T cell clones may not possess the ability to lyse tumor. Anichini et al., [Anichini A. et al., J. Immunol. 156, 208-17 (1996)] have shown that many CTL respond to as-yet uncharacterized antigens. In this situation, less refined methods have been used to stimulate CTL, such as tumor lysates, eluted peptides, tumor cell/DC fusions, or genetically modified tumor cells. However, the major disadvantage of these methods is the fact that they all are being basically empirical. In addition, in these situations it is expected that only a small percent of recognized proteins will be critical to the anti-tumor CTL response.

In addition to CD8⁺ T cells, which are stimulated by peptides presented in the context of MHC-class I, other CD8⁺ cells, such as natural-killer-cytotoxic T lymphocytes (NK-CTL), present antigens in the context of HLA-E [Moretta L. et al., Trends Immunol. 24, 136-43 (2003)]. Other CD8⁺ cells are stimulated by lipid antigens in the context of MHC class I-like molecules, such as CD1d [Vincent M S. et al., Nat. Immunol. 4, 517-23 (2003)].

CTL that recognize and take up TAA, in addition to their value in the identification and characterization of new TAA, have been recently demonstrated as an effective tool for cell immunotherapy in metastatic melanoma patients. Adoptive cell therapy has recently shown extremely encouraging results. S. A. Rosenberg and his group treated 35 patients with metastatic malignant melanoma by infusion of Tumor Infiltrating Lymphocytes (TILs) cultures. Eighteen (51%) of 35 treated patients experienced objective clinical responses including three ongoing complete responses [Dudley M E. et al., J. Clin. Oncol. 23, 2346-57 (2005)]. Sites of regression included metastases to lung, liver, lymph nodes, brain, and cutaneous and subcutaneous tissues. This dramatic response has never been reported before. However, major problems of this approach are (a) the need to expand TILs 1000-2000-fold, a process that is unaffordable, and (b) the fact that many of the infused cells are irrelevant, non cytotoxic and what is worse—of regulatory suppressive function.

The ideal ligand for clonal T cells labeling and detection technique would be the TCR. In fact, T cell labeling by TCR-specificity is already at hand. These are the peptide-MHC complexes built as tetramers. Tetramer use is restricted to pre-determined HLA alleles and known peptides of these restrictions. Within this limit, tetramers are an excellent tool to determine T cell frequencies by flow cytometry and their functional characterization [Denkberg G. et al., Eur. J. Immunol. 30, 3522-32 (2000)]. Tetramers have also been employed for T cell cloning and selection in the clinical context, although to a very limited extent [Cobbold M. et al., JEM 202, 379-386 (2005)]. The huge diversity of TCRs makes it impractical to produce tetramers for each and every T cell clone. Large amounts of tetramers are costly and expensive. But even if that aspect is overlooked, the major problem with using tetramers to sort for tumor reactive T cells is the dissociation of antigen specificity from functional cytotoxicity [Echchakir H. et al., Proc. Natl. Acad. Sci. USA 99, 9358-63 (2002)]. Tetramer positive cells do not necessarily lyse tumor targets.

An alternative to the selection of T cells by their antigen specificity is to label them through activation markers, i.e. cytokine production. Two methods have been devised that allow decorating cells according to their cytokine profile. One is the intracellular cytokine staining. Using this method, the cells have to be fixed and permeated for staining, and therefore are not available for further use. A second method is cytokine capture. This second method is useful to isolate live T cells, but slight diversion from optimal working conditions may sort irrelevant cells because of their proximity to diffused cytokine molecules. Therefore, using these methods the main problem, selecting for functional cytotoxic antigen-reactive T cells, still remains.

A similar approach, detection of T cells following their stimulation, is based on mechanical fusion of lysosomal-associated membrane glycoproteins (LAMPs) to the plasma membrane of an effector cell once it has undergone degranulation [Rubio, V. et al., Nat. Med. 9, 1377-1382 (2003)]. With the advent of antibodies specific for LAMP, including CD107a, this process can be observed using flow cytometry. This methodology is the first to select T cells based on functional reactivity. However, a stimulus that activates T cells may trigger degranulation just as it may trigger cytokine release. Thus, activated T cell may be sorted not based on antigenic specificity.

TCR is a member of the receptor tyrosine kinase family. Typical to these receptors is to rapidly internalize together with their soluble ligands. As the ligand of TCR, the pMHC, is anchored within the target cell membrane, researchers were prompted to look for a mechanistic explanation on how the TCR:pMHC interaction occurs. In 2001, Hudrisier et al. reported that following sustained TCR signaling, CTLs promptly capture target cell membranes together with the antigenic peptide as well as various other surface proteins. By using antigenic peptides and lipids that were fluorescently labeled, they demonstrated that the mechanism by which pMHC complexes are acquired by the T cell is through capture of plasma membrane fragments [Hudrisier D. et al., J. Immunol. 166, 3645-9 (2001)]. They further showed that membrane capture by CTL correlates tightly with their lytic function. Their observation recapitulated the pioneering publication of [Huang J F. et al., Science 286, 952-4 (1999)] and [Stinchcombe J C. et al., Immunity. 15, 751-61 (2001)].

The inventors of the present invention thus hypothesized that if indeed T cells are capable of capturing membrane from target cells, labeling of the target would eventually lead to labeling of the T cell reactive to it. Furthermore, labeled CD8+T cells will be the ones involved in tumor killing and can be selectively isolated. The following Examples demonstrate that these assumptions were indeed validated.

The present invention therefore shows that the melanoma-derived gp100 protein, when loaded on DC from melanoma patients, stimulated autologous CD8⁺ lymphocytes. These lymphocytes were found in close contact with dead melanoma cells and contained membrane material transferred from stained melanoma cells, as demonstrated by direct visualization. However, in cultures containing control lymphocytes stimulated with unloaded DC, no melanoma cell killing was observed. The data of the present invention further indicate clearly that these CTL have the ability to kill autologous target tumor cells and therefore may be used for adoptive transfer.

As indicated above, currently, isolation of specific T cells and production of T cell lines is a relatively complicated and prolonged procedure, based on presentation of known peptides by APC. The present invention provides a method for rapid and simple isolation of tumor-specific T cells. The method of the invention is based on the fact that CTL rapidly capture membrane fragments from target cells in a T cell receptor (TCR) signaling-dependent manner. The method of the invention involves co-incubation of membrane-labeled target cells with lymphocytes, followed by cell sorting of lymphocytes after membrane transfer from labeled targets has taken place. These lymphocytes are then propagated in vitro for large-scale production of antigen-specific lymphocytes, for example, tumor-specific lymphocytes for cancer immunotherapy.

Moreover, T cell selection based on their cytotoxic activity is a unique and original tool that enables the isolation and expansion of the most important T cell sub-population: those cells that actually destroy the tumor cells. These cells cannot be sorted directly by any other known methodology. Using the membrane capture technology, there is no need for preliminary knowledge on tumor associated antigens, the method is not limited to specific HLA alleles, and a variety of cells may be used as targets. A major application is for improved adoptive cell therapy.

The technology provided by the present invention represents a flexible tool to be employed for diseases with known, and in particular with unknown antigenic repertoire. It should be noted that the same principle is also applicable for the treatment of autoimmune and infectious diseases.

Another important application of CTL labeling and sorting based on membrane capture is for analysis of the immune status (immunomonitoring) in the setting of cancer vaccination or otherwise elicited immunity. This tool may also be useful in research of tumor associated antigens that mediate tumor regression.

It is therefore one object of the invention to provide a rapid and simple method for the isolation of CD8⁺ tumor-specific T cells, based on membrane transfer from labeled targets to lymphocytes. This method is specifically applicable for the isolation and characterization of antigen-specific CD8⁺ T lymphocytes, and to the large-scale production of tumor-specific lymphocytes for cancer immunotherapy, particularly, by adoptive transfer.

Another object of the invention is to provide a composition comprising T cells reactive to a pathologic disorders, and methods for the treatment of such disorders.

These and other objects of the invention will become apparent as the description proceeds.

### SUMMARY OF THE INVENTION

In a first aspect the invention relates to a method for the preparation and isolation of an immune system cell, preferably, a lymphocyte, which is capable of specifically recognizing an antigen related to a pathologic disorder. This method comprises the steps of: (a) providing target cells expressing or presenting an antigen related to said pathologic disorder, or any fragment or peptide thereof; (b) labeling said target cells with a suitable membrane detectable label; (c) providing a sample comprising immune system cells, preferably, the sample may be obtained from a subject suffering from said pathologic disorder; (d) co-incubating the labeled target cells obtained in step (b) with the cells, preferably, lymphocytes provided in step (c) for a suitable period of time under suitable conditions; (e) identifying a population of immune system cells, preferably, lymphocytes expressing the CD8⁺ antigen, from the co-incubated cells obtained in step (d), by a suitable means such as addition of specific antibody; (f) selecting from the CD8⁺ antigen expressing cells identified in step (e) cells stained with said membrane-label using suitable means under suitable conditions, wherein said selected cells are CD8⁺/membrane-label double positive cells; (g) propagating the CD8⁺/membrane-label double positive cells selected in step (f), under suitable conditions; and (h) evaluating the biological activity of the cells obtained in step (g) on an end-point indication using a suitable test system, whereby difference in said end point compared to a suitable control is indicative of the biological activity of said cells.

According to one embodiment, the method of the invention is intended for the isolation of lymphocytes, specifically, CTL (Cytotoxic T Lymphocytes) capable of recognizing an antigen specific for a pathologic disorder.

According to a second aspect, the invention relates to a pharmaceutical composition for the treatment of a pathologic disorder. The composition of the invention comprises as an active ingredient a lymphocyte capable of recognizing an antigen specific for said pathologic disorder, and optionally further comprises pharmaceutically acceptable additive, carrier, excipient, stabilizer, and any other therapeutic constituents.

According to a specifically preferred embodiment, the lymphocyte comprised within the composition of the invention may be a CTL (Cytotoxic T Lymphocyte). Preferably, such lymphocyte may be obtained by the method of the invention.

Specifically preferred compositions are particularly suitable for the treatment of any malignant disorder, specifically, melanoma.

The invention further provides a method for the treatment of a pathologic disorder in a subject in need thereof, comprising the step of administering to said subject a therapeutically effective amount of a lymphocyte capable of recognizing an antigen specific for said pathologic disorder or of a composition comprising the same, preferably, the compositions of the invention.

According to a specifically preferred embodiment, the lymphocyte used by the method of the invention may be obtained as described by the invention.

The invention further relates to the use of a lymphocyte capable of recognizing an antigen specific for a pathologic disorder in the preparation of a pharmaceutical composition for the treatment of said pathologic disorder. Preferably, such lymphocyte may be obtained by the method defined by the invention.

### BRIEF DESCRIPTION OF THE INVENTION

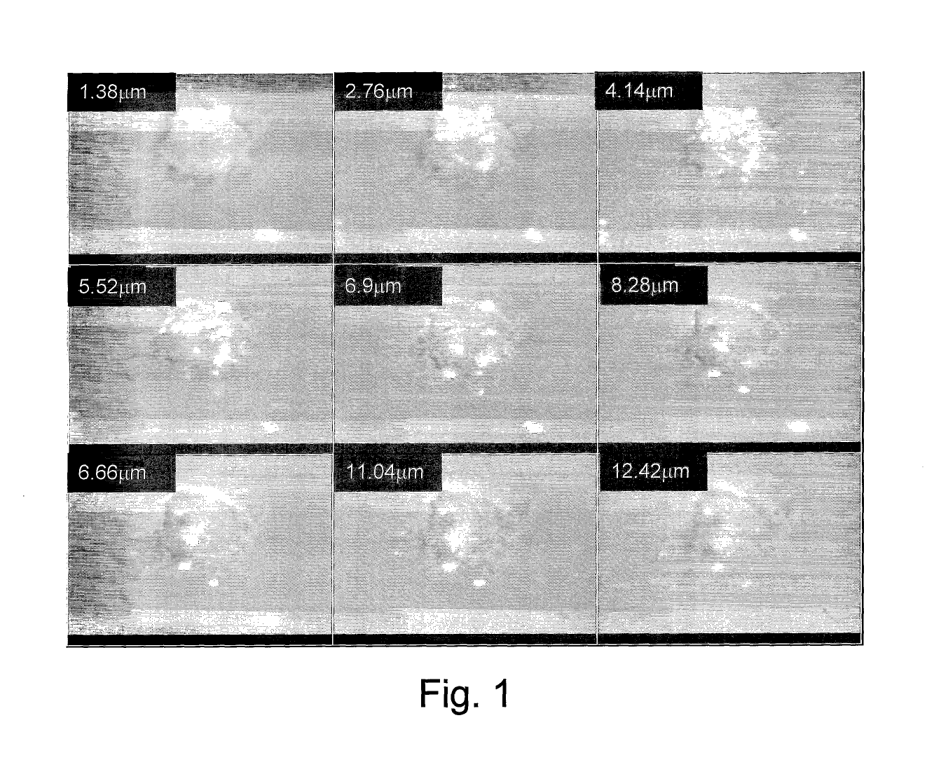

FIG. 1 Confocal microscopy of dendritic cells taking-up FITC-labeled HR-gp100

Confocal microscopy photographs showing that FITC-labeled HR-gp100 protein is taken up and found inside dendritic cells (maximal uptake at 3-11 μm depth).

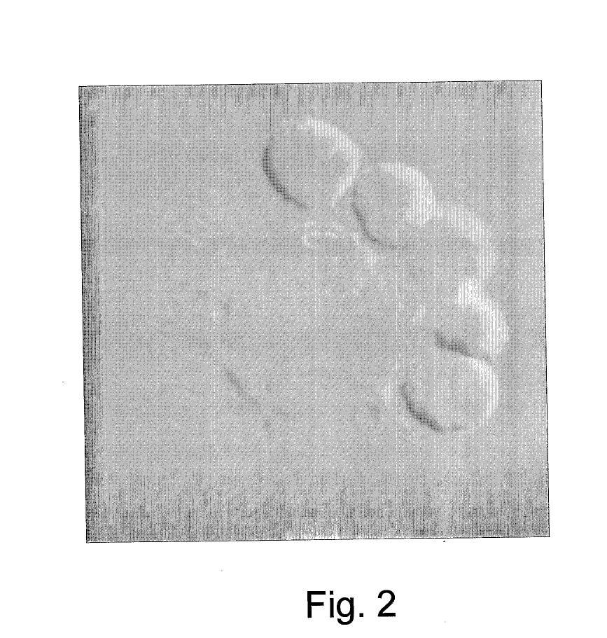

FIG. 2 Confocal microscopy of interaction between dendritic cell loaded with HR-gp100 and autologous CD8⁺ lymphocytes

Confocal microscopy photograph showing interaction of a dendritic cell loaded with HR-gp100 and autologous CD8⁺ lymphocytes obtained from a melanoma patient. Lens ×40/1.3 oil digital zoom 8.0

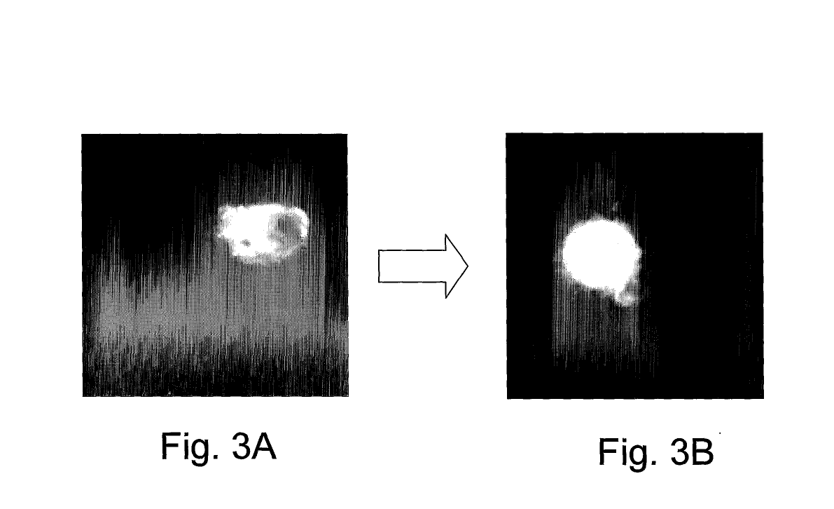

FIG. 3A-3B Co-incubation of CD8⁺ lymphocytes from a melanoma patient and a DiD- and CMFDA-stained autologous melanoma cell

FIG. 3A: an unstained lymphocyte can be seen in the vicinity of a stained melanoma cell. Zoom 3.3

FIG. 3B: attachment of a lymphocyte to a melanoma cell induced transfer of membranal stain from a melanoma cell to an autologous CD8⁺ lymphocyte.

Zoom 5.6

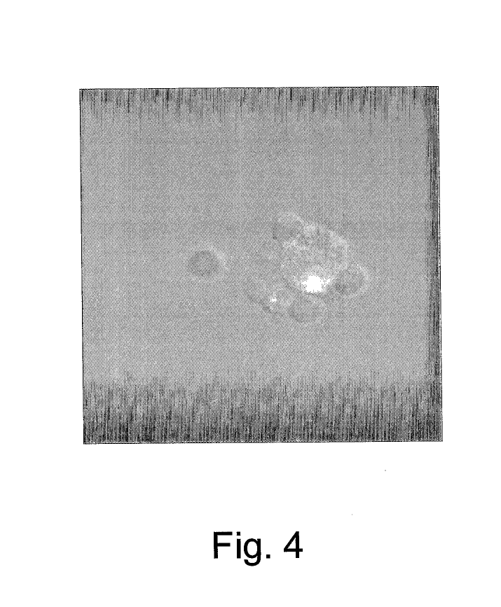

FIG. 4 Confocal microscopy photograph of DiD-labeled melanoma cell

Confocal microscopy photograph demonstrating a DiD-labeled melanoma cell which shows signs of destruction after attachment to autologous CD8⁺ lymphocytes, previously activated with HR-gp100-loaded DC. Zoom 3.3

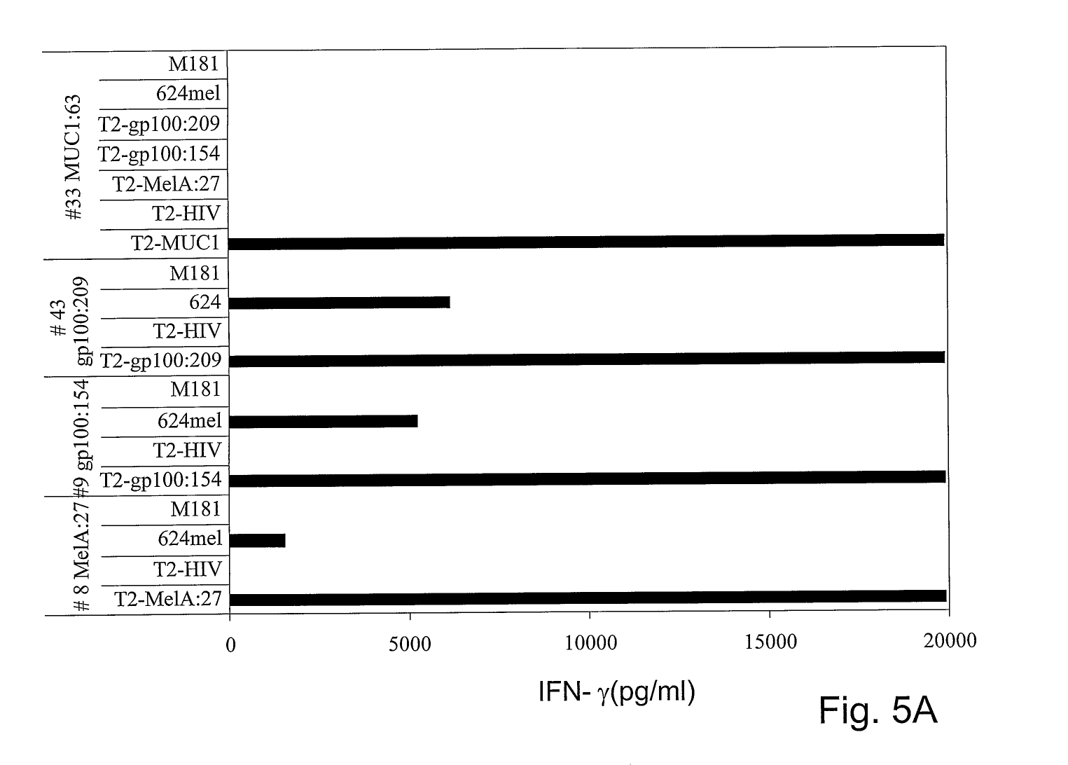

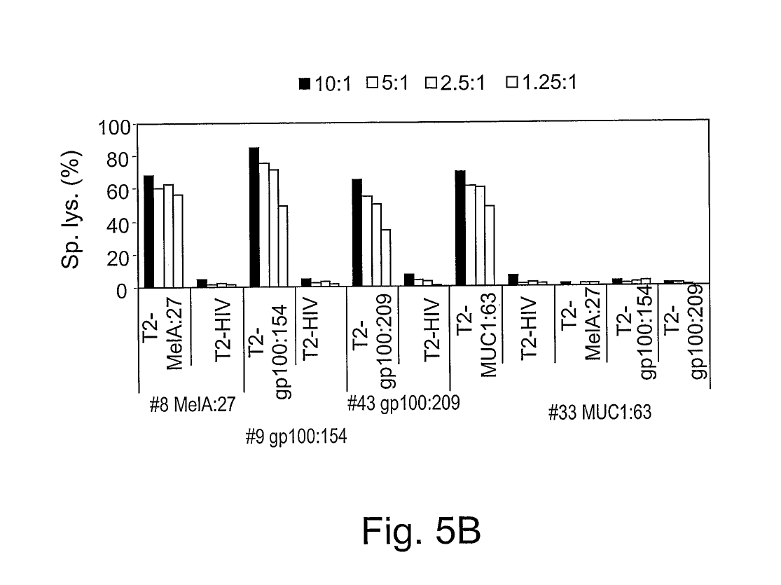

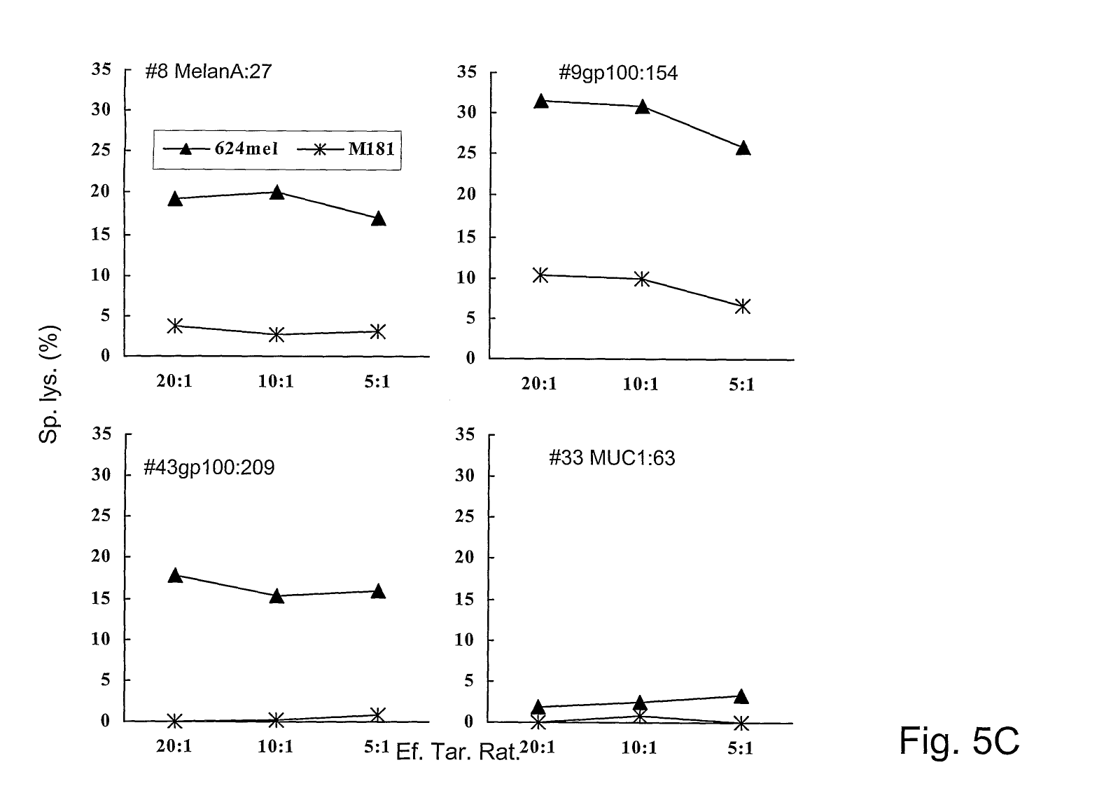

FIG. 5A-5C Reactivity of peptide specific T lymphocytes

FIG. 5A: shows IFN-γ (interferon gamma) secretion in pg/ml (picogram per milliliter) in 20 hours co-culture supernatants of the four different specific T cell-lines of the invention, L-8, L-9, L-43 and L-33, which were co-cultured with T2 cells preloaded with 1 μM correspondent or control peptide or HLA-A*0201-positive (624mel) versus HLA-A*0201-negative (M181) tumor cells.

FIG. 5B: shows cytotoxic activity of peptide-specific T lymphocytes. The lymphocytes (effector cells) were admixed at different ratios (10:1 to 1.25:1) with 5×10³ [³⁵S]-L-L-methionine labeled peptide-loaded T2 cells as targets.

FIG. 5C: shows cytotoxic activity of peptide-specific T lymphocytes. The lymphocytes (effector cells) were admixed at different ratios (20:1 to 5:1) with 5×10³ [³⁵S]-L-methionine labeled tumor cells. Percentage of specific lysis was calculated as follows: % lysis=(cpm in experimental well−cpm spontaneous release)/(cpm maximal release−cpm spontaneous release)×100. Abbreviations: sp. Lys. (specific lysis), Ef. Tar. Rat. (effector to target ratio).

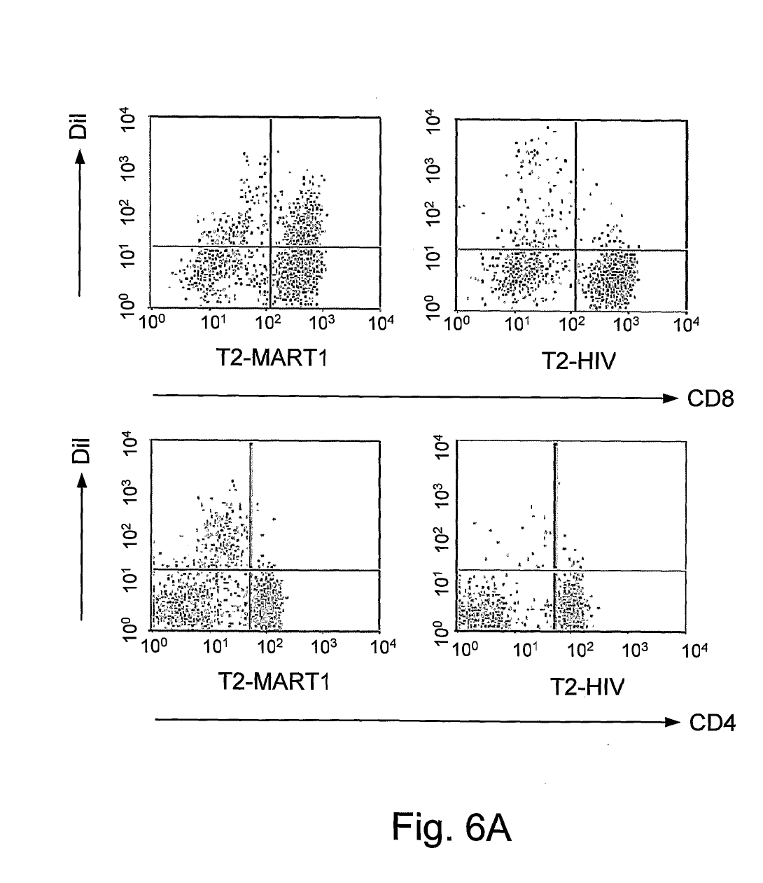

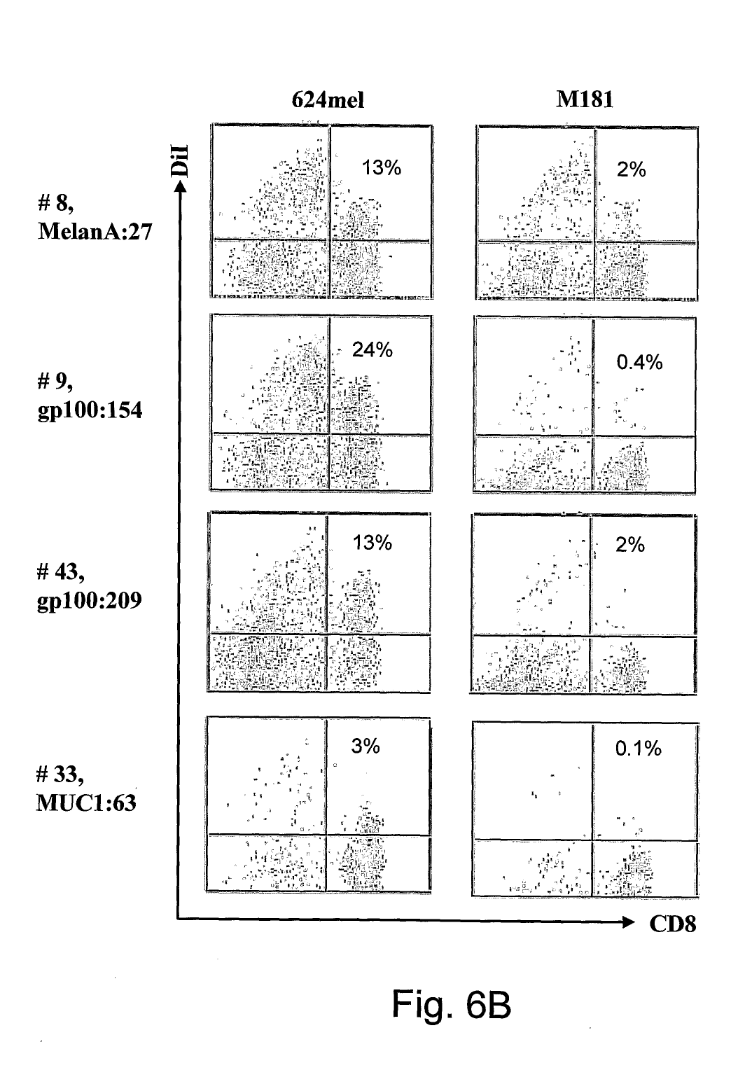

FIG. 6A-6B Specific capture of target cell membrane fragments by melanoma antigen peptide-reactive CD8⁺ T lymphocytes

FIG. 6A: shows flow cytometry analysis of the Specific T cell line L-8, of the invention. DiI-stained T2 cells pulsed with 1 μM of MelanA/Mart1:27 or HIV-derived peptide were co-cultured with MelanA/Mart1:27-specific T cells (L-8). Following incubation at 37° C. for 1 h, cells were washed, stained with FITC-conjugated anti-CD8 (left panel) or anti-CD4 (right panel) mAb's and subjected to flow cytometry. The upper right quadrant represents the CD8⁺ (CD4)DiI⁺ double positive population.

FIG. 6B: shows flow cytometry analysis of the four different specific T cell lines of the invention L-8, L-9, L-43 and L-33 (as control). DiI-stained 624mel or M181 melanoma cells were co-cultured with MelanA/Mart1:27 (L-8), gp100:154 (L-9), gp100:209 (L-43) or MUC1:63 (L-33)-reactive T lymphocytes. After incubation at 37° C. for 4 h, cells were washed, stained with FITC-conjugated anti-CD8 mAb and analyzed by flow cytometry.

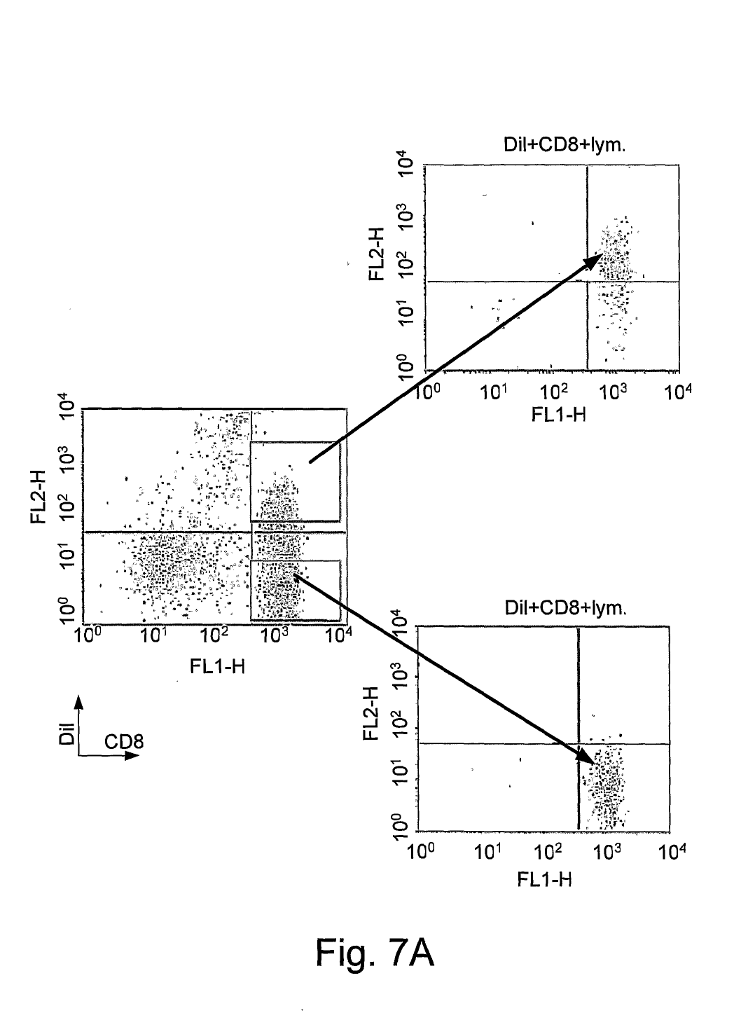

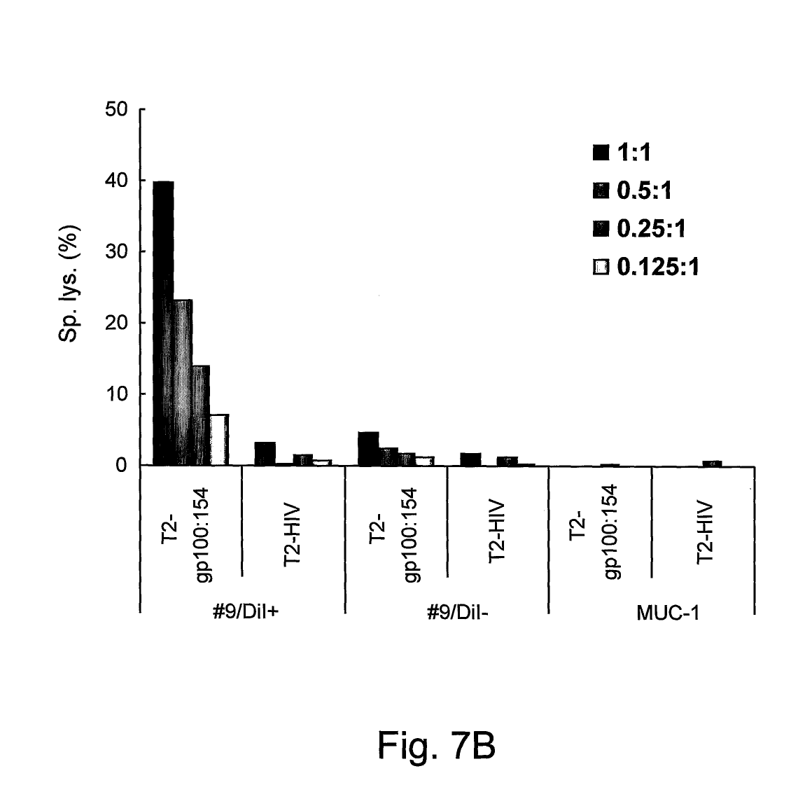

FIG. 7A-7B Comparative analysis of cytotoxic activity of DiI⁺ versus DiI⁻ CD8 lymphocytes

FIG. 7A: DiI staining of gp100:154-reactive CD8⁺ lymphocytes before selection and in positive and negative fractions. DiI-stained 624mel melanoma cells were co-cultured with gp100:154-specific lymphocytes (line L-9). Following incubation at 37° C. for 4 h, cells were washed, stained with FITC-conjugated anti-CD8 mAb and separated by FACS into positive (upper right) and negative (lower right) fractions according to DiI staining.

FIG. 7B: Cytotoxic activity of DiI⁺ and DiI⁻ CD8 lymphocytes. The separated cells were expanded in vitro by rapid expansion protocol and examined in 5 h [³⁵S]□Methionine□release CTL assay with gp100:154-loaded T2 cells as specific targets. The HIV-loaded T2 cells were used as irrelevant targets. The effector-to-target ratios of 1:1 to 0.125:1 are shown. Abbreviations: Sp. Lys. (specific lysis), Lym (lymphocytes).

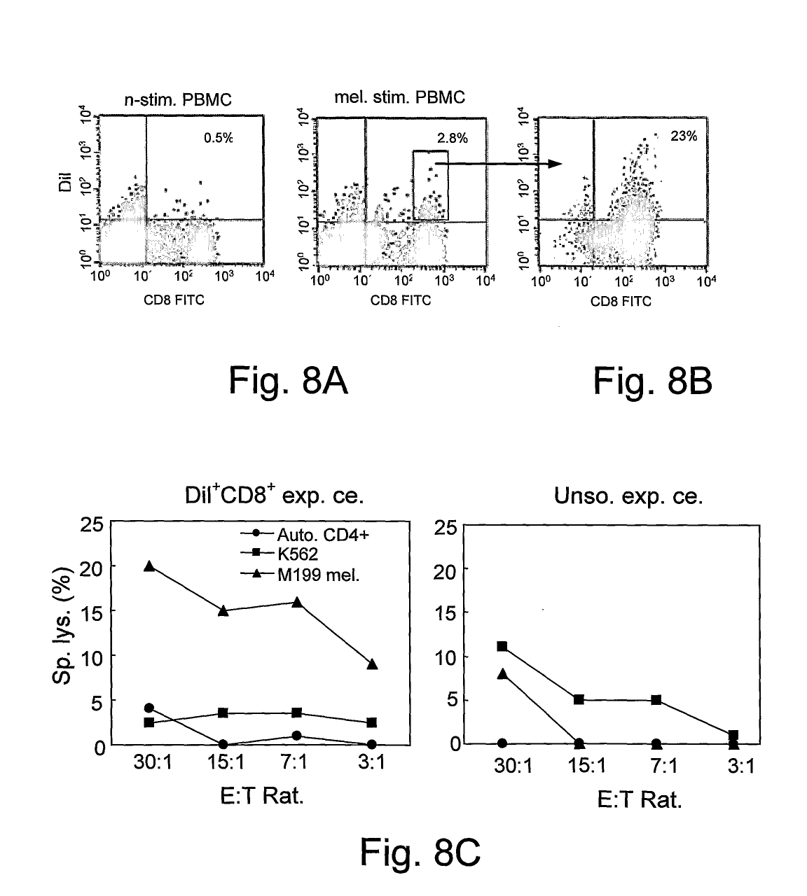

FIG. 8A-8C Isolation, expansion and functional characterization of DiI⁺CD8⁺ lymphocytes from bulk PBMC

FIG. 8A: shows flow cytometry of stimulated versus non-stimulated PBMC. PBMC were stimulated by irradiated (17000 rad) M199 autologous melanoma cells in complete medium supplemented with 300 IU/ml IL-2 (right panel) or were cultured in absence of melanoma cells (left panel). After 12 days, the bulk cultures were harvested and co-incubated with DiI-stained autologous melanoma cells at 37° C. for 6 h, stained by anti-CD8 mAb and analyzed by flow cytometry.

FIG. 8B: shows flow cytometry of stimulated and expanded PBMC. DiI⁺CD8⁺ cells were isolated by FACS and expanded for 12 days using rapid expansion protocol. Following expansion, the cells were co-incubated with DiI-stained M199 melanoma cells and analyzed by flow cytometry.

FIG. 8C shows comparison of the cytotoxic activity of lymphocytes sorted and expanded from PBMC, versus the activity of unsorted expanded cells. DiI⁺CD8⁺ (left panel) and unsorted (right panel) expanded cells were tested in CTL assays against M199 as specific and K562 and autologous CD4⁺ cells, as non-specific targets (left panel). The effector-to-target ratios of 30:1 to 3:1 are shown. Abbreviations: Sp. Lys. (specific lysis), n. stim. (non-stimulated), mel. (melanoma), stim. (stimulated), exp. (expanded) ce. (cells), Unso. (unsorted), Rat. (ratio), Auto (autologous).

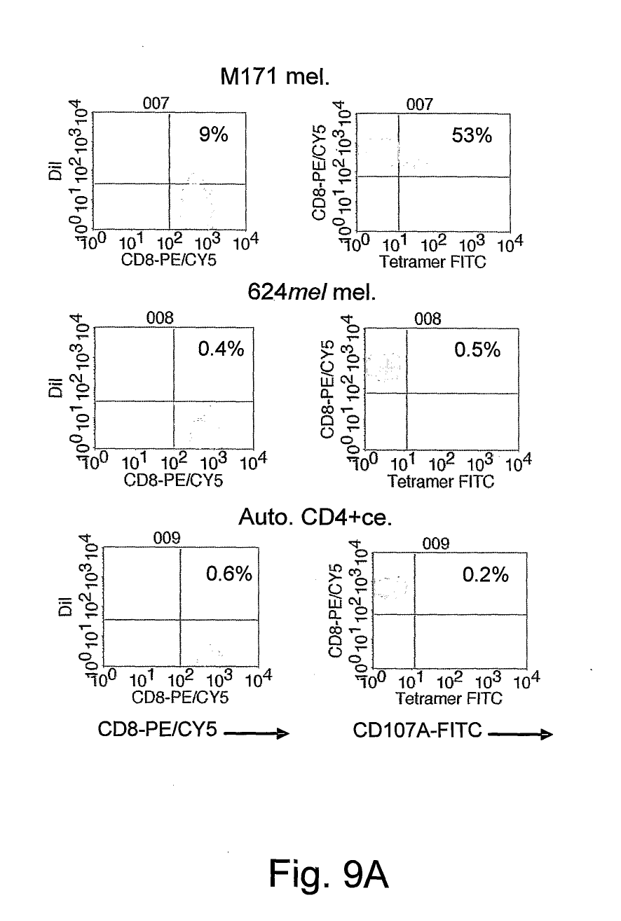

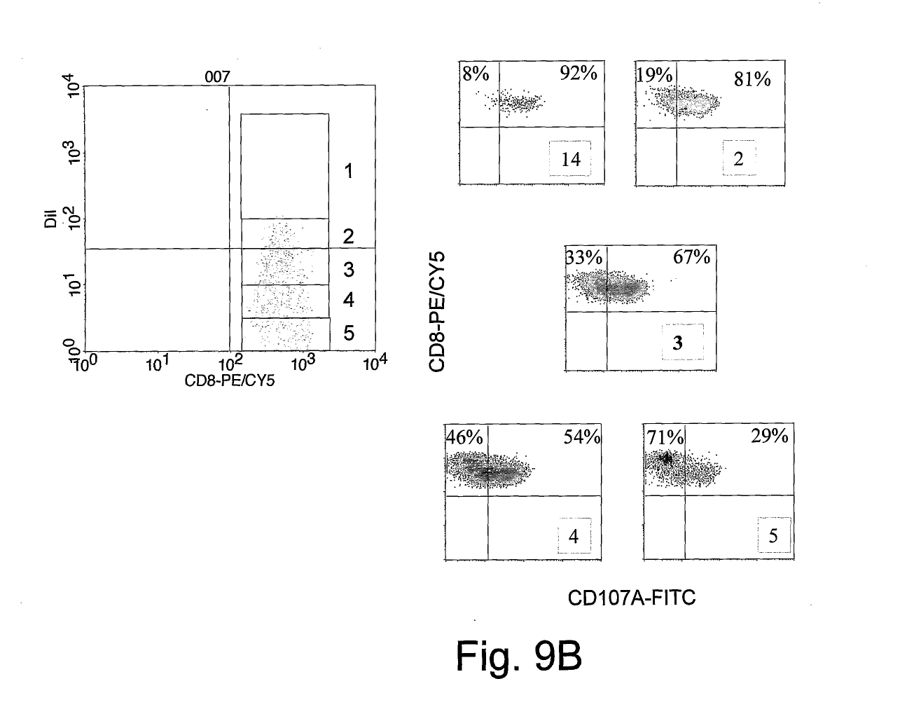

FIG. 9A-9B Acquisition of target cell membrane fragments by CD8⁺ lymphocytes is correlated with CD107A mobilization

The M-171 patient-derived CD8⁺ lymphocytes were stimulated for 12-days by irradiated (17000 rad) autologous melanoma cell in complete medium supplemented with 300 IU/ml IL-2.

FIG. 9A: The stimulated lymphocytes were incubated with DiI-stained autologous M-171 melanoma and analyzed for CD107A mobilization by flow cytometry. HLA-mismatched 624mel melanoma cells and autologous CD4⁺ lymphocytes served as irrelevant targets. Lymphocytes were gated on CD8⁺ cells and the percentages of CD8⁺ DiI⁺ (left column) and CD8⁺CD107A⁺ (right column) double positive cells are indicated.

FIG. 9B: Following incubation with DiI-stained M-171 cells, the CD8⁺ gated lymphocytes were further subgated according to DiI staining, from CD8⁺DiI^{high} (1,2) through CD8⁺ DiI^{intermediate} (3) to CD8⁺ DiI^{low} (4,5) and analyzed for CD107A expression. The percentage of CD8⁺ CD107A⁺ and CD8⁺ CD107A⁻ cells are indicated (upper right and upper left quadrants, respectively).

### DETAILED DESCRIPTION OF THE INVENTION

A number of methods of the art of molecular biology are not detailed herein, as they are well known to the person of skill in the art. Such methods include site-directed mutagenesis, PCR cloning, expression of cDNAs, analysis of recombinant proteins or peptides, transformation of bacterial and yeast cells, transfection of mammalian cells, and the like. Textbooks describing such methods are e.g., Sambrook et al., Molecular Cloning A Laboratory Manual, Cold Spring Harbor Laboratory; ISBN: 0879693096, 1989, Current Protocols in Molecular Biology, by F. M. Ausubel, ISBN: 047150338X, John Wiley & Sons, Inc. 1988, and Short Protocols in Molecular Biology, by F. M. Ausubel et al., (eds.) 3rd ed. John Wiley & Sons; ISBN: 0471137812, 1995. These publications are incorporated herein in their entirety by reference, including publications cited therein. Furthermore, a number of immunological techniques are not in each instance described herein in detail, as they are well known to the person of skill in the art. See e.g., Current Protocols in Immunology, Coligan et al., (eds), John Wiley & Sons. Inc., New York, N.Y.

Conventional chemotherapy aims at controlling the growth of cancer such as melanoma by targeting rapidly growing cells. However, this function is not specific, as many normal cells, like those of the bone marrow and the intestinal epithelium, also have a basal level of proliferation. Therefore, many normal cells of the body also are susceptible to the toxic effects of chemotherapy, and conventional chemotherapy may have a substantial negative impact on the patient.

Immunotherapy is a specific protocol and is therefore attractive. If antigens were expressed on the tumor cells but were not expressed by normal cells of the host, then specific cytolytic T lymphocytes could theoretically be activated to selectively kill the tumor cells while sparing the normal tissue of the patient. To this end, considerable effort has been made in the last decade to identify such tumor specific antigens, which may serve as targets for specific tumor cell killing.

Immune recognition of these antigens occurs via specific CD8⁺ CTL (cytotoxic T lymphocytes) that interacts with antigenic peptides bound to a groove in MHC Class I (HLA) molecules. MHC Class II-binding epitopes recognized by CD4⁺ T cells have also been described. Under optimal circumstances, initiation of an immune response is triggered by peptide presented by the MHC complexes expressed by host APC, and additionally requires multiple cofactors provided by APC. After initial activation, CTL induced by APC interactions are thought to migrate throughout the host, recognize the same MHC/peptide complex in the tumor cells, and be triggered to kill them. This antigen-specific cytolysis is mediated largely via induction of apoptosis.

The present invention describes a novel approach of the preparation, isolation and the enrichment of a specific CD8⁺ lymphocyte population which recognizes specific antigen present in a particular pathologic disorder. The method of the invention is based on membrane capture technology, which enables isolation of CTL's targeted against unknown antigenic targets specific for a certain pathologic disorder. This, without any prior knowledge of such antigen, and without any HLA limitations.

Thus, in a first aspect, the invention relates to a method for the preparation and isolation of an immune system cell, preferably, a lymphocyte, which is capable of specifically recognizing an antigen related to a pathologic disorder. It should be appreciated that said specific recognition may be preferably mediated by MHC Class I molecules. This method comprises the steps of: (a) providing target cells expressing or presenting an antigen related to said pathologic disorder, or any fragment or peptide thereof; (b) labeling said target cells with a suitable membrane detectable label; (c) providing a sample comprising immune system cells, preferably, lymphocytes, more preferably, the sample may be obtained from a subject suffering from said pathologic disorder; (d) co-incubating the labeled target cells obtained in step (b) with the cells, preferably, lymphocytes provided in step (c) for a suitable period of time under suitable conditions; (e) identifying a population of immune system cells, preferably, lymphocytes expressing the CD8⁺ antigen, from the co-incubated cells obtained in step (d), by a suitable means such as addition of specific antibody; (f) selecting from the CD8⁺ antigen expressing cells identified in step (e) cells stained with said membrane-label using suitable means under suitable conditions, wherein said selected cells are CD8⁺/membrane-label double positive cells; (g) propagating the CD8⁺/membrane-label double positive cells selected in step (f), under suitable conditions; and (h) evaluating the biological activity of the cells obtained in step (g) on an end-point indication using a suitable test system, whereby difference in said end point compared to a suitable control is indicative of the biological activity of said cells. As a non-limiting example, it should be noted that appropriate control may be CD4⁺/membrane-label double positive cells, CD8⁺ cells (for example, DiI-CD8⁺ lymphocytes), unsorted CD8⁺ lymphocytes (effector cell control) and stimulation by non-relevant target.

According to one embodiment, the method of the invention is intended for the isolation of lymphocytes, specifically CTL (Cytotoxic T Lymphocytes) capable of recognizing an antigen which is specific for a pathologic disorder.

According to a specifically preferred embodiment, the lymphocytes provided for the method of the invention are autologous PBMC (peripheral blood mononuclear cells) obtained from a subject suffering from the same pathologic disorder. These lymphocytes will be educated by the method of the invention to specifically recognize an antigen related to said disorder, and will be subsequently selected and propagated.

In another preferred embodiment, the target cells used by the method of the invention may be cells obtained from a subject suffering from said pathologic disorder.

The pathologic disorder may preferably be an immune-related disorder, such as a malignant disorder, pathologic viral or bacterial infection, an inflammatory disorder and an autoimmune disorder.

According to a specific embodiment, the pathologic disorder may be a malignant disorder such as melanoma, carcinoma, leukemia, sarcoma and lymphoma. More specifically, the malignant disorder may be melanoma.

In a particular and preferred embodiment, the target cell used by the method of the invention may be a tumor cell obtained from a primary tumor or a tumor cell line. In yet another embodiment, the target cell may be a bacterial or virus infected cell.

In an alternative particular embodiment, the target cell used by the method of the invention may be an antigen presenting cell (APC) expressing or presenting the pathologic disorder-related antigen. It should be noted that the APCs serving as target cells by the method of the invention may express or present any fragment or peptide derived from such antigen. Several cell types appear to be capable of serving as APC, including dendritic cells (DC), activated B cells, T2 cells (TAP-deficient lymphoblastoid cells line) and activated macrophages. In accordance with the invention the APCs are preferably autologous cells and in some illustrative embodiments the antigen-presenting cell may be a dendritic cell (DC). It is understood that one of skill in the art will recognize that other antigen presenting cells may be useful in the invention, such as B cells activated by lipopolysaccharide, whole spleen cells, peripheral blood macrophages, fibroblasts or non-fractionated peripheral blood mononuclear cells (PBMC). Therefore, the invention is not limited to the exemplary cell types which are specifically mentioned and exemplified herein.

In order to obtain APCs from a subject, particularly human patients, blood is drawn from the patient by cytopheresis, a procedure by which a large number of white cells is obtained, while other blood components are being simultaneously returned back to the patient. The target cells used by the method of the invention may be prepared from these cells and frozen in small aliquots.

Accordingly, where APC is used as a target cell by the method of the invention, such cell may be loaded or transfected with an antigen specific for the pathologic disorder. The antigen may be provided for the purpose of loading APCs, in any form. For example, a peptide, a purified recombinant protein, a fusion protein, a nucleic acid construct encoding for said antigen, a cell lysate, supernatant or any preparation of a host cell expressing said construct, a cell line and tissue endogenously expressing said antigen.

Preparation of cell lysates as well as loading or pulsing into dendritic cells may be performed in variety ways. Different procedures of lysate loading to DC may lead to enhancement of antigen presentation, or to the specific stimulation of a certain type of immune response. It is therefore appreciated that lysates and any antigen preparation as well as loading procedures may be performed in different ways.

As indicated above, the antigen may be provided to the APCs as a nucleic acid construct. As used herein, the term “nucleic acid” refers to polynucleotides such as deoxyribonucleic acid (DNA), and, where appropriate, ribonucleic acid (RNA). The terms should also be understood to include, as equivalents, analogs of either RNA or DNA made from nucleotide analogs, and, as applicable to the embodiment being described, single-stranded and double-stranded polynucleotides. “Construct”, as used herein, encompasses vectors such as plasmids, viruses, bacteriophage, integratable DNA fragments, and other vehicles, which enable the integration of DNA fragments into the genome of the host.

Expression vectors are typically self-replicating DNA or RNA constructs containing the desired gene or its fragments, and operably linked genetic control elements that are recognized in a suitable host cell and effect expression of the desired genes. These control elements are capable of effecting expression within a suitable host. Generally, the genetic control elements can include a prokaryotic promoter system or a eukaryotic promoter expression control system. This typically includes a transcriptional promoter, an optional operator to control the onset of transcription, transcription enhancers to elevate the level of RNA expression, a sequence that encodes a suitable ribosome binding site, RNA splice junctions, sequences that terminate transcription and translation and so forth. Expression vectors usually contain an origin of replication that allows the vector to replicate independently of the host cell.

A vector may additionally include appropriate restriction sites, antibiotic resistance or other markers for selection of vector-containing cells. Plasmids are the most commonly used form of vector but other forms of vectors which serve an equivalent function and which are, or become, known in the art are suitable for use herein. See, e.g., Pouwels et al., Cloning Vectors: a Laboratory Manual (1985 and supplements), Elsevier, N.Y.; and Rodriquez, et al. (eds.) Vectors: a Survey of Molecular Cloning Vectors and their Uses, Buttersworth, Boston, Mass (1988), which are incorporated herein by reference.

It should be further noted that APCs which, according to a particular embodiment of the invention serve as target cells by the method of the invention, may be loaded with cell lysates or any other preparations of host cells which express the particular antigen.

“Host cell” as used herein refers to cells which can be recombinantly transformed with vectors constructed using recombinant DNA techniques. A drug resistance or other selectable marker is intended in part to facilitate the selection of the transformants. Additionally, the presence of a selectable marker, such as drug resistance marker may be of use in keeping contaminating microorganisms from multiplying in the culture medium. Such a pure culture of the transformed host cell would be obtained by culturing the cells under conditions which require the induced phenotype for survival.

It should be noted that any fusion protein, for example, any particular antigen or any fragment and peptide thereof and a tag sequence, may also be used for the preparation of APCs as target cells and therefore is contemplated within the scope of the invention. Tag sequences may include, but are not limited to GFP (green fluorescent protein), GST (glutathione-S-transferase), myc, Flag, His6 and HA. By “fusion protein” or alternatively, “chimeric protein”, is meant a fusion of a first amino acid sequence encoding any antigen specific to a particular pathologic disorder, with a second amino acid sequence defining a domain foreign to and not substantially homologous with any domain of the subject.

An “antigen” is a molecule or a portion of a molecule capable of being bound by an antibody, which is additionally capable of inducing an animal to produce antibody capable of binding to an epitope of that antigen. An antigen may have one or more than one epitope. The specific reaction referred to above is meant to indicate that the antigen will react, in a highly selective manner, with its corresponding antibody and not with the multitude of other antibodies which may be evoked by other antigens. The term “epitope” is meant to refer to that portion of any molecule capable of being bound by an antibody that can also be recognized by that antibody. Epitopes or “antigenic determinants” usually consist of chemically active surface groupings of molecules such as amino acids or sugar side chains, and have specific three-dimensional structural characteristics as well as specific charge characteristics.

As indicated herein-above, the method of the invention enables isolation of CTL's specific for known or unknown antigens related to a pathologic disorder. According to a specific embodiment, the malignant disorder may be melanoma. In such case, as a non-limiting example for known melanoma associated antigens, the target antigen may be any one of the known melanoma-associated antigens (MAA) tyrosinase, gp-100, MAGE-3 and MART-1, or any combination thereof.

The isolation of the specific antigen-directed lymphocytes of the invention is based on their membrane-capture properties. Therefore, the target cell must be labeled with any appropriate detectable label which enables interaction between the target and the lymphocyte and detection of the desired lymphocyte. Therefore, any membrane-label may be used. As a non-limiting example, the target cells may labeled by 1,1′-dioctadecyl-3,3,3′,3′-tetramethylindocarbocyanine perchlorate (DiI) or 1,1-dioctadecyl-3,3,3′,3′-tetramethyl-indodicarbocyanine 4-chlorobenzene-sulfonate salt (DiD). It should be noted that both labels were used by the invention, as shown in the following Examples.

It should be further appreciated that the detectable label may further contain a further compound acting as a tag for facilitating the isolation of the desired TCL's.

An additional cell labeling method may be based on the use of new lipophilic tags to improve the efficiency of T cell sorting, and a capture device for the selection of cytotoxic T cells tagged with the lipophilic reagents

According to one preferred embodiment, suitable means for identifying CD8⁺ expressing cells according to step (e) is by addition of an antibody specific for CD8. Such antibody specifically binds and thereby stains cells expressing CD8⁺. According to another preferred embodiment, the anti-CD8⁺ antibody used by the method of the invention for selecting CD8⁺ lymphocytes may be any one of a polyclonal and a monoclonal antibody, preferably, an anti CD8⁺FITC monoclonal antibody.

Monoclonal antibodies may be prepared from B cells taken from the spleen or lymph nodes of immunized animals, in particular rats or mice, by fusion with immortalized B cells under conditions which favor the growth of hybrid cells.

The technique of generating monoclonal antibodies is described in many articles and textbooks, such as the above-noted Chapter 2 of Current Protocols in Immunology. Spleen or lymph node cells of these animals may be used in the same way as spleen or lymph node cells of protein-immunized animals, for the generation of monoclonal antibodies as described in Chapter 2 therein. The techniques used in generating monoclonal antibodies are further described by Kohler and Milstein [Nature 256; 495-497, (1975)], and in U.S. Pat. No. 4,376,110.

The term “antibody” is meant to include intact molecules as well as fragments thereof, such as, for example, Fab and F(ab′)₂, which are capable of binding antigen.

It will be appreciated that Fab and F(ab′)₂ and other fragments of the antibodies are within the scope of the present invention and may be used for the compositions and the methods disclosed herein for intact antibody molecules. Such fragments are typically produced by proteolytic cleavage, using enzymes such as papain (to produce Fab fragments) or pepsin (to produce F(ab′)₂ fragments).

It should be noted that the antibody used by the method of the invention may be directly or indirectly labeled, by using a secondary antibody. One of the ways in which an antibody in accordance with the present invention can be detectably labeled is by linking the same to an enzyme. This enzyme, in turn, when later exposed to an appropriate substrate, will react with the substrate in such a manner as to produce a chemical moiety which can be detected, for example, by spectrophotometric, fluorometric or by visual means. Enzymes which can be used to detectably label the antibody include, but are not limited to, malate dehydrogenase, staphylococcal nuclease, delta-5-steroid isomerase, yeast alcohol dehydrogenase, α-glycerophosphate dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholin-esterase. The detection can be accomplished by colorimetric methods, which employ a chromogenic substrate for the enzyme. Detection may also be accomplished by visual comparison of the extent of enzymatic reaction of a substrate in comparison with similarly prepared standards.

It is also possible to label an antibody in accordance with the present invention with a fluorescent compound, fluorescence emitting metals, a chemi-luminescent compound or a bioluminescent compound.

According to a preferred embodiment, suitable means for selection of cells double stained with CD8⁺ and said membrane-label according to step (f) of the method of the invention may be FACS analysis, preferably, under sterile conditions.

According to the method of the invention, cells selected as CD8⁺ cells containing the target-cell label are propagated under suitable conditions. Suitable conditions for propagating the selected cells may be as a non-limiting example, any addition of cytokines and growth factors selected from the group consisting of cytokines, such as IL-2 and IL-15, antibodies, such as OKT3, or lectins such as phytohemagglutinin, that lead to non-specific stimulation of lymphocytes, and/or the incubation of the selected cells in the presence of a feeder cell layer, preferably, irradiated PBMC.

The selected propagated cells obtained by the method of the invention are subsequently evaluated for their biological activity. According to one embodiment, suitable test system for evaluating the biologic activity of propagated selected cells may be in vitro/ex-vivo cell culture or in-vivo animal model. According to one specifically preferred embodiment, the test system used of evaluation may be an in vitro/ex-vivo cell culture of said antigen specific immune system cell, preferably, the isolated lymphocytes of the invention. Suitable means for evaluating the biologic activity of propagated selected cells may be for example the in vitro stimulation of lymphocytes with the target cells for 24-48 hours, and determination of cytokine production, for example, IFN-γ, IL-5 and IL-10 by a suitable means, preferably, a protein based detection assay, for example, flow cytometry, ELISA and ELISPOT assays, most preferably, by ELISA.

As a non-limiting example, a suitable means for quantitation of IFN-γ, IL-5 and IL-10 may be ELISA. ELISA is an acronym for Enzyme-Linked ImmunoSorbent Assay. A quantitative method for estimating the amount of a compound using antibodies linked to an enzyme that catalyses an easily measurable color reaction.

Alternatively, or additionally, an in vitro stimulation of lymphocytes with the tumor target cells and measurement of cytotoxic activity by a suitable means, for example, using ³⁵S-methionine labeled target cells may be used for evaluation [as detailed in Carmon L. et al., J. Clin. Invest. 110, 453-62 (2002)].

Alternatively, the lymphocytes isolated by the method of the invention may be evaluated using as a preferred test system, an in vivo animal model of CD1^{nu/nu} nude mice. As exemplified in Example 9, the feasibility of said isolated cells as suitable for adoptive transfer may be evaluated by examining as an end point indication, their ability to inhibit tumor growth and increase mice survival, as compared to a suitable control. As a non-limiting example, it should be noted that appropriate control may be CD4⁺/membrane-label double positive cells, DiI-CD8⁺ lymphocytes and unsorted CD8⁺ lymphocytes.

The present invention therefore further provides a cytotoxic T cell lymphocyte (CTL) specific for an antigen related to a pathologic disorder. The CTL of the invention may preferably be isolated by the method of the invention.

According to a second aspect, the invention relates to a pharmaceutical composition for the treatment of a pathologic disorder. The composition of the invention comprises as an active ingredient a lymphocyte capable of recognizing an antigen specific for said pathologic disorder, and optionally further comprises at least one pharmaceutically acceptable additive, carrier, excipient and/or stabilizer, and may further comprise other therapeutic constituents.

According to a specifically preferred embodiment, the lymphocyte comprised within the composition of the invention may be a CTL (Cytotoxic T Lymphocyte). Preferably, such lymphocyte may be obtained by the method of the invention.

According to a specifically preferred embodiment, the composition of the invention is specifically applicable for the treatment of a pathologic disorder such as for example, a malignant disorder, pathologic viral or bacterial infection, an inflammatory disorder and an autoimmune disorder.

Preferred pharmaceutical compositions of the invention are particularly intended for the treatment of a malignant disorder such as, carcinoma, melanoma, leukemia, sarcoma and lymphoma. For example, prostate, ovary, kidney, lung, brain, breast, colon, bone, skin, testes and uterus cancer may be treated.

As used herein to describe the present invention, “cancer”, “tumor” and “malignancy” all relate equivalently to a hyperplasia of a tissue or organ. If the tissue is a part of the lymphatic or immune systems, malignant cells may include non-solid tumors of circulating cells. Malignancies of other tissues or organs may produce solid tumors. In general, the composition and the methods of the present invention may be used in the treatment of non-solid and solid tumors, and for monitoring and imaging of solid tumors.

Specifically preferred compositions are particularly suitable for the treatment of melanoma.

The term melanoma includes, but is not limited to, melanoma, metastatic melanoma, melanoma derived from either melanocytes or melanocyte-related nevus cells, melanocarcinoma, melanoepithelioma, melanosarcoma, melanoma in situ, superficial spreading melanoma, nodular melanoma, lentigo maligna melanoma, acral lentiginoous melanoma, invasive melanoma or familial atypical mole and melanoma (FAM-M) syndrome. Such melanomas may be caused by chromosomal abnormalities, degenerative growth and developmental disorders, mitogenic agents, ultraviolet radiation (UV), viral infections, inappropriate tissue gene expression, alterations in gene expression, or carcinogenic agents. The aforementioned melanomas can be treated by the method and the composition described in the present invention.

Alternatively, the compositions and methods of the present invention may be directed to cells that are infected by pathogenic viruses such as HIV, EBV, CMV, Vaccinia, MVM, ECMV, Herpes or Influenza virus.

The compositions of the invention are particularly intended for the induction of immune response in a mammalian subject, preferably, in humans, but other mammals including, but not limited to, monkeys, equines, cattle, canines, felines, mice, rats, pigs, horses, sheep and goats may be treated.

The pharmaceutical composition used by the methods of the invention can be prepared in dosage unit forms and may be prepared by any of the methods well-known in the art of pharmacy. In addition, the pharmaceutical compositions used by the invention may further comprise pharmaceutically acceptable additives such as pharmaceutical acceptable carrier, excipient or stabilizer, and optionally other therapeutic constituents. Naturally, the acceptable carriers, excipients or stabilizers are non-toxic to recipients at the dosages and concentrations employed.

The compositions of the present invention may be administered directly to the subject to be treated or it may be desirable to conjugate them to carrier proteins such as ovalbumin or serum albumin prior to their administration. Therapeutic formulations may be administered in any conventional dosage formulation. Formulations typically comprise at least one active ingredient, as defined above, together with one or more acceptable carriers thereof.

Composition dosages may be any that induce an immune response. It is understood by the skilled artisan that the preferred dosage would be individualized to the patient following good laboratory practices and standard medical practices.

Each carrier should be both pharmaceutically and physiologically acceptable in the sense of being compatible with the other ingredients and not injurious to the patient. While formulations include those suitable for oral, rectal, nasal, preferred formulations are intended for parenteral administration, including intramuscular, intravenous, intradermal and specifically subcutaneous administration. The formulations may conveniently be presented in unit dosage form and may be prepared by any methods known in the art of pharmacy.

The compositions of the invention can be administered in a variety of ways. By way of non-limiting example, the composition may be delivered intravenously, or into a body cavity adjacent to the location of a solid tumor, such as the intraperitoneal cavity, or injected directly into or adjacent to a solid tumor. Intravenous administration, for example, is advantageous in the treatment of leukemias, lymphomas, and comparable malignancies of the lymphatic system.

As a preferred route the composition of the present invention may be administered via subcutaneous or intradermal injections in proximity to the tumor, via intralymphatic or intravenous injection.

The pharmaceutical forms suitable for injection use include sterile aqueous solutions or dispersions and sterile powders for the extemporaneous preparation of sterile injectable solutions or dispersions. In all cases the form must be sterile and must be fluid to the extent that easy syringeability exists. It must be stable under the conditions of manufacture and storage and must be preserved against the contaminating action of microorganisms, such as bacteria and fungi. The carrier can be solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants.

The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above.

In the case of sterile powders for the preparation of the sterile injectable solutions, the preferred method of preparation are vacuum-drying and freeze drying techniques which yield a powder of the active ingredient plus any additional desired ingredient from a previously sterile-filtered solution thereof.

As used herein “pharmaceutically acceptable carrier” includes any and all solvents, dispersion media, coatings, antibacterial and antifungal agents and the like. The use of such media and agents for pharmaceutical active substances is well known in the art. Except as any conventional media or agent is incompatible with the active ingredient, its use in the therapeutic composition is contemplated.

Supplementary active ingredients can also be incorporated into the compositions.

Although it is not envisioned as a preferred route, the composition of the invention or its active ingredients, the antigen specific CD8⁺ lymphocytes, may also be orally administered, for example, with an inert diluent or with an assimilable carrier, or enclosed in hard or soft shell gelatin capsule, or compressed into tablets, or incorporated directly with the food of the diet. The invention further provides a method for the treatment of a pathologic disorder in a subject in need thereof, comprising the step of administering to said subject a therapeutically effective amount of a lymphocyte capable of recognizing an antigen specific for said pathologic disorder or of a composition comprising the same, preferably, the compositions of the invention.

As used herein, “effective amount” means an amount necessary to achieve a selected result. For example, an effective amount of the composition of the invention useful for the treatment of said pathology.

The method of the invention is particularly useful in the treatment of carcinomas, lymphomas, melanomas and sarcomas, more preferably melanomas.

It should be noted that the method of the invention may employ any of the compositions of the invention. According to a specifically preferred embodiment, the lymphocyte used by the method of the invention may be obtained as described by the invention

In a preferred embodiment, the method of the invention is intended for treating a mammalian subject, preferably, a human. Therefore, by “patient” or “subject in need” is meant any mammal for which immunotherapy is desired, including human bovine, equine, canine, and feline subjects, preferably, human patient.

As used herein in the specification and in the claims section below, the term “treat” or treating and their derivatives includes substantially inhibiting, slowing or reversing the progression of a condition associated with a certain pathologic disorder, substantially ameliorating clinical symptoms of a condition or substantially preventing the appearance of clinical symptoms of a condition or disorder.

The invention further relates to the use of a lymphocyte capable of recognizing an antigen specific for a pathologic disorder in the preparation of a pharmaceutical composition for the treatment of said pathologic disorder.

Still further, the invention provides a method for preparing a therapeutic composition for the treatment of a pathologic disorder in a subject in need thereof. This method comprises the steps of: (a) isolating a cytotoxic T cell lymphocyte specific for an antigen related to said pathologic disorder, preferably, by the isolation method described by the invention; and (b) admixing the isolated lymphocyte with at least one of a pharmaceutical acceptable carrier, diluent, excipient and/or additive.

As described by Example 4, it should be further appreciated that the invention also provides for T cell lines that were generated from metastatic melanoma HLA-A*0201-positive patients that specifically recognized peptides from gp100 and Melan A/MART1. More particularly, as a further aspect, the invention relates to the following T cell lines L-8, specific for Melan-A/MART1:27-35 (AAGIGILTV, also denoted by SEQ ID NO:4), L-9 for gp100:154-162 (KTWGQYWQV, also denoted by SEQ ID NO:3), L-43 for gp100:209-217 (210M, IMDQVPFSV, also denoted by SEQ ID NO:2) and L-33 for MUC1:63-71.

In yet another aspect, the invention relates to a further important application of CTL labeling and sorting by the method of the invention, which is based on membrane capture. This aspect relates to the possible analysis of an immune status (immunomonitoring) in the setting of cancer vaccination or otherwise elicited immunity. This tool may also be useful in research of tumor associated antigens that mediate tumor regression.

Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present invention will be limited only by the appended claims and equivalents thereof.

As used in the specification and the appended claims and in accordance with long-standing patent law practice, the singular forms “a” “an” and “the” generally mean “at least one”, “one or more”, and other plural references unless the context clearly dictates otherwise. Thus, for example “a cell”, “a peptide” and “an antigen” include mixture of cells, one or more peptides and a plurality of antigens of the type described.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The contents of all publications quoted herein are fully incorporated by reference.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

### Experimental Procedures

### Reagents

### Antibodies:

*antiCD8⁺ and anti CD4⁺
*anti CD107A

### Cell Labeling

*DiI(1,1′-dioctadecyl-3,3,3′,3′-tetramethylindocarbocyanine perchlorate), *1,1-dioctadecyl-3,3,3′,3′-tetramethyl-indodicarbocyanine 4-chlorobenzene-sulfonate salt (DiD), a lipophilic tracer for membrane staining, and 5-chloromethylfluorescein diacetate (CMFDA), a green fluorescence cytoplasma stain, were purchased from Molecular Probes.

### Cytokines:

rIL-2—recombinant IL-2 was purchased from Chiron Co.

### TNF, 1000 U/ml, R&D Systems

### Cell-Medium and Buffers

*FCS—Fetal calf serum (Biology Industries, Beth Haemek, Israel);
*CM (complete medium) contains the following: RPMI 1640, 2 mM glutamine, 20 μg/ml gentamycin, 10 mM hepes, 1 mM non-essential amino acids, 1 mM sodium pyruvate (Biological Industries, Beth Haemek, Israel).
*BD IMag buffer: Phosphate Buffered Saline (PBS), 0.5% Bovine Serum Albumin (BSA), (Sigma) and 2 mM EDTA.

### Methods:

### Patients

Peripheral blood mononuclear cells (PBMC) were from melanoma patients who had participated in a clinical trial of post-operative adjuvant administration of an autologous melanoma vaccine. The vaccine consisted of 8 doses of autologous irradiated and dinitrophenyl-modified cultured melanoma cells, administered every 21-28 days, as described [Lotem (2002) ibid.]. All patients included in the present study developed a strong delayed type hypersensitivity response to their melanoma cells. In some cases, frozen blood from healthy donors was used.

Preparation of Mature HR-gp100-Loaded Dendritic Cells

Mononuclear cells (MNC) were incubated at 37° C. in RPMI-1640 medium containing 10 mM hepes, 1 mM non-essential amino acids, 1 mM sodium pyruvate, 2 mM glutamine, 100 μg/ml streptomycin, 100 U/ml penicillin (all from Biological Industries, Beth Haemek, Israel) (complete medium, CM) and 2% autologous human serum, at a concentration of 4×10⁶/ml/well (24-well plates, Nunc, Denmark). After 90 minutes, the nonadherent (lymphoid) cells were collected and cryopreserved in liquid N₂ in cryovials, each containing 20-30×10⁶ cells in 10% DMSO and 20% FCS, for further use as responding cells (see below). The adherent cells were further grown in 1 ml CM in the presence of 1000 U/ml granulocyte macrophage-colony stimulating factor (GM-CSF, Leucomax, Sandoz, Switzerland) and 500 U/ml IL-4 (R&D Systems). IL-4 was added once at day 3 or 4. At day 5 or 6, the cells had the characteristics of immature DC, with few (usually <1%) lymphocytes. The DC were washed and incubated in the original wells in 1 ml of serum-free CM, in the presence of IL-4 and HR-gp100 (10-100 μg/ml, Gelbart Y. et al., Protein Expression and Purification (2004)). After 4 hours, 2% serum and maturation factors were added, and the cells incubated for 2 more days. The maturation factors were tumor necrosis factor-α (TNF, 1000 U/ml, R&D Systems), and prostaglandin E2 (PG, 1 μM, Sigma) Rieser C. et al., J. Exp. Med. 186, 1603-8 (1997); Jonuleit H. et al., Eur. J. Immunol. 27, 3135-42 (1997)].

### Labeling of Melanoma Cells

Melanoma cell lines were grown from biopsies obtained from patients, as previously described [Lotem M. et al., Br. J. Cancer 86, 1534-9 (2002)]. Cells were labeled according to the manufacturer's protocol.

### Co-Incubation of Melanoma Cells and Autologous PBMC

Melanoma cells from Melanoma patients were provided by The Sharett Institute of Oncology at Hadassah Hospital. The melanoma cells were thawed in a 37° C. water bath and suspended in 10 ml RPMI 1640 (Gibco) containing 10% FCS in 50 ml tube. The tube was centrifuged for 7 minutes at 1100 rpm. The supernatant was discarded and the cells were suspended in 1 ml RPMI. A 13 μl sample was taken from the cells to a 96 U shaped well plate and diluted with 13 μl trypan blue (Sigma) and counted in a hemocytometer (Marienfield). For proliferation, 2×10⁵ melanoma cells were transferred to 10 ml RPMI containing 10% FCS in a 25 cm² tissue culture flask, and incubated at 37° C. and 5% CO₂. Nine ml of RPMI containing 10% FCS were added to the remaining 8×10⁵ and irradiated at 17,000 rad. After irradiation, the melanoma cells were centrifuged at 1100 rpm. The supernatant was discarded and the cells were resuspended in 5 ml CM containing 10% AB serum and transferred to a well in a 6 well plate (Nunc) and incubated at 37° C. Serum was obtained from the Blood Bank in Tel Hashomer and was inactivated in a 56° C. water bath for 30 minutes before adding to the CM. After adding the serum, the CM was filtered through a 0.454 filter.

Autologous PBMC were thawed in a 37° C. water bath and suspended in 10 ml RPMI 1640 containing 10% FCS in 50 ml tube. The tube was centrifuged for 7 minutes at 1100 rpm. The supernatant was discarded and the cells were suspended in 1 ml RPMI. A 13 μl sample was taken from the cells to a 96 U shaped well plate and diluted with 13 μl trypan blue and counted using hemocytometer. Approximately, 8×10⁶ PBMC were transferred with five ml CM containing 10% AB to melanoma cells containing well for co-incubation, total volume of 10 ml. Another 8×10⁶ PBMC were transferred with 10 ml CM containing 10% AB to a well in a 6 well plate for control. The remaining 6×10⁷ PBMC were frozen.

On day 3,200 U/ml IL-2 (Chiron, U.S.A) was added to each well. On day 5, 5 ml out of 10 ml supernatant were discarded, and fresh CM containing 10% B was added along with 50 U/ml IL-2 to each well. The same routine as on day 5 was performed on day seven. On day 9, CD8⁺ TL cells were separated by anti-human CD8 magnetic particles (BD™) according to the manufacturers instructions. Briefly, cells from all the wells were transferred to 50 ml tubes and centrifuged at 1100 rpm. The supernatant was discarded and the cells were washed with 10 ml sterile BD IMag buffer. Cells were centrifuged at 1100 rpm and the supernatant was discarded. The anti-human CD8 magnetic particles were thoroughly pipetted and 50 μl were added to 10μ cells, mixed thoroughly and transferred to 5 ml polystyrene round-bottom tubes for 30 minutes incubation at room temperature. One ml of the BD IMag buffer was added to the cell suspension and the tubes were immediately placed on the BD™ IMagnet for 10 minutes incubation at room temperature. While the tubes were placed on magnet, the supernatant which contains the CD8− negative fraction was carefully aspirated and discarded. Tubes were removed, 1 ml of BD Imag buffer was added to the cells, and after gentle pipetting the tubes were returned to the magnet for another 4 minutes. Supernatant was discarded while the tubes were on the magnet. The tubes were removed, 1 ml of BD Imag buffer was added to the cells, and after gentle pipetting the tubes were returned to the magnet for another 4 minutes. The supernatant was discarded and the positive CD8 cells were resuspended in 1 ml CM containing 10% AB and counted. 1.5×10⁵ CD8⁺ cells were separated from the PBMC originated from the co-incubation well, and 5×10⁵ CD8⁺ cells were separated from the PBMC originated from the control well. The cells were transferred to a 24 well plate in 2 ml CM containing 10% AB with 200 U/ml IL-2 and incubated at 37° C.

Active Membrane Transfer Labeled with DiI from Melanoma to Activated CTL

About 4×10⁵ melanoma cells were divided to two 50 ml tubes and washed twice with 1 ml RPMI. After the second washing, the supernatant was discarded and the melanoma cells were resuspended in 1 ml RPMI containing 10% FCS and 511 DiI. The melanoma cells were incubated at 37° C. for 30 minutes. After incubation the cells were washed five times with 1 ml RPMI. After the fifth wash, the supernatant was discarded and the cells were resuspended in 1 ml CM containing 10% AB and transferred to a 24 well plate. Approximately 2×10⁶ CD8⁺ lymphocytes from the co-incubation well in 1 ml of CM containing 10% AB were added to one of the wells containing DiI stained-melanoma, and 2×10⁶ CD8⁺ from the control well in 1 ml CM containing 10% AB were added to the other DiI stained-melanoma well. The cells were co-incubated for 10 hours at 37° C.

### Sorting of Double Labeled CTL

Cells from the co-incubation wells were harvested, transferred to 50 ml tubes and washed twice with 1 ml sterile PBS. Supernatant was discarded, and cells were resuspended in 200 μl sterile PBS containing 40 μl fluoroscein isothiocyanate (FITC) anti-human CD8 (5:1 ratio). As a positive control, 3×10⁵ CD8⁺ were resuspended in 100 μl sterile PBS containing 20 μl FITC anti-human CD8. As an isotype control 3×10⁵ CD8⁺ were resuspended in 100 μl sterile PBS containing 20 μl FITC mouse IgG1. Tubes were incubated for 30 minutes on ice. Cells were washed twice with 1 ml sterile PBS and resuspended in 1 ml sterile PBS. Cell-suspensions were filtered through a 15 μl filter in to sterile 5 ml polystyrene round-bottom tubes. Cells, positive for anti CD8 FITC and DiI (CD8⁺ DiI⁺), and cells positive only for anti CD8 FITC (CD8⁺ DiI⁻) were sorted out by the FACscan sorter into CM containing 50% AB with Gentamycin (200 μg/ml) and pen′-strep′ nystatin (1000 units/ml penicillin, 1 mg/ml streptomycin and 125 units/ml nystatin) in 5 ml round-bottom tubes. During sorting process, sorted cells were counted by the FACscan sorter and incubated at 37° C. for 2 hours recovery.

### 12-Day Rapid Expansion of Sorted CTL

Sorted cells were incubated on a platform of irradiated PBMC as feeder cells.

Feeder cells: About 2×10⁷ PBMC were thawed and resuspended in 20 ml RPMI containing 10% FCS and irradiated at 4000 rad. The cells were washed once with 1 ml RPMI, resuspended in 10 ml CM containing 10% AB and were divided in to two wells in a 6 well plate, each well containing 1×10⁶ feeder cells in 5 ml.

Sorted cells (CD8⁺ DiI⁺ and CD8⁺ DiI⁻), were centrifuged and the supernatant was discarded. The cells were resuspended in 5 ml CM containing 10% AB, and were transferred to the wells containing irradiated feeder cells to a total volume of 10 ml. Gentamycin and pen′-strep′ nystatin were added to prevent contamination.

For rapid expansion 30 ng/ml OKT3-murine monoclonal antibody for CD3 (Ortho biotech inc.) were added. The cells were incubated at 37° C. On day 2, 5 ml of the supernatant were aspirated and 5 ml of fresh CM containing 100 U/ml IL-2 was added. The same routine was performed on days 4, 6, 8 and 10.

### IFN-γ Release by Melanoma-Stimulated CD8⁺ T Cells

Approximately 1×10⁵ of CD8⁺ DiI⁺ and of CD8⁺ DiI⁻ cells were incubated with about 1×10⁴ autologous melanoma cells overnight in 250 μl CM +10% AB, at a ratio of 10:1 in a 96 well plate. After the co-incubation, 100 μl supernatant were aspirated and tested for IFN-γ release in an ELISA assay with Diaclone reagents according to the manufacturer's protocol. Plates were coated with monoclonal anti-human IFN-γ and kept overnight in the 4° C. Plates were washed and PBS+5% BSA was added, and incubated for blocking the non-bound sites at room temperature for 2 hours. After blocking, plates were washed 3 times and dried by tapping the plate upside down on an absorbent paper. Supernatant samples from the co-incubation wells were added to the ELISA test plate along with biotynilated anti-human IFN-γ detection antibody for 2 hours at room temperature. After 3 washes, HRP-Strep (Horse Radish Peroxidase conjugated Streptavidin) was added for 20 minutes incubation at room temperature. After 3 washes, TMB (3,3′,5,5′-tetramethylbenzidine) substrate solution was added. Sulfuric acid (H₂SO₄) was added to stop color development. The reading was performed by an ELISA reader at 450 nm.

### ³⁵[S]-Methionine Release Cytotoxicity Assay

CD8⁺ DiI⁺, and CD8⁺ DiI⁻ effector cells were incubated with each of the target cells. The effector: target ratios examined were: 30:1, 15:1, 7.5:1, 3.25:1. 3.5×10⁵ target cells of each cells type were washed twice with sterile PBS×1 and resuspended in 0.5 ml methionine-free RPMI (Sigma) containing 10% FCS and 2.5 μl ³⁵[S]-methionine (Redivue PRO-MIX) containing 25μ Ci. The cells were incubated at 37° C. for 4 hours. After 4 hours, the ³⁵[S]-methionine-labeled cells were washed once with 5 ml PBS. 3,000 cells of each target cells type were distributed as mentioned with 100 μl CM containing 10% AB into the co-incubation wells to a total volume of 200 μl. For ³⁵S methionine release calibration, the methionine-labeled target cells were distributed to 12 wells, 3,000 each in 100 μl CM containing 10% AB. 6 wells for spontaneous ³⁵[S]-methionine release and 6 wells for total ³⁵[S]-methionine release. The plates were incubated at 37° C. for 5 hours. 15 minutes prior to the completion of incubation, 100 μl NaOH-0.1N were added to the total ³⁵[S]-Methionine release wells, and 100 μl CM containing 10% AB were added to the spontaneous ³⁵[S]-methionine release wells. After the 5 hours incubation the plates were centrifuged for 5 minutes in 1000 rpm. 50 μl supernatant were aspirated and transferred to a β-counter microplate containing 150 μl scintillation liquid (Microscint™ 40) and kept overnight at 4° C. covered in aluminum foil. ³⁵[S]-Methionine release counts were measured in a microplate scintillation β-counter (Packard). Specific killing equation: [cpmEXP−cpmSPONT)/(cpmTOTAL−cpmSPONT)]×100=Specific killing %. To rule out non specific killing by NK cells, NK sensitive K-562 cell line was used as control target cells. CD4⁺ cells were used as an autologous control target. CD8⁺ DiI⁻ were used as effector cells control.

### Isolation and Expansion of Functional Tumor-Specific CD8⁺ Lymphocytes for Adoptive Transfer

The following melanoma antigen peptide-reactive CD8 T lines are used: L-43, reactive to gp100:_{209-217(210M)} (IMDQVPFSV, also denoted by SEQ ID NO:2); L-9, reactive to gp100:₁₅₄₋₁₆₂ (KTWGQYWQV, also denoted by SEQ ID NO:3); L-8, reactive to Melan-A/MART1:₂₇₋₃₅ (AAGIGILTV, also denoted by SEQ ID NO:4). 624mel melanoma cells are stained with DiI according to the manufacturer's instructions, washed five times and co-cultured in 24-well plates (2 ml complete medium/well) with 1-2×10⁶ effector cells at E:T ratio of 3:1. Following incubation for 5 h, cells are washed and stained with anti-CD8 mAb (Ebioscience). DiI⁺ and the DiI⁻ CD8⁺ positive lymphocytes are sorted out by flow cytometry. Following extensive washing in PBS the lymphocytes are re-suspended in lymphocyte medium at 10⁶ cells/ml. The cells are seeded in 24-well plates pre-coated with anti-CD3 mAb and incubated for 72 hours at 37° C. in 5% CO₂. The cells are then be expanded further in the presence of IL-2 (100 U/ml).

### 624mel Melanoma Model

Male CD1^{nu/nu} mice receiving 4 Gy total body irradiation (TBI) from a 137Cs source, followed by subcutaneous (s.c.) inoculation in the upper back of 1×10⁶ 624mel (HLA-A*0201⁺ gp100⁺ MART-1⁺) melanoma cells admixed with Matrigel (BD PharMingen, San Diego, Calif.). Seven days later, 5×10⁶ CD8⁺ T cells are injected around the tumors. The adoptive transfer is followed by local s.c. administration of 1000 U rhIL-2 twice a day for 5 days. Tumor size is measured in two perpendicular dimensions three times per week. Mice are sacrificed once tumors reached 400 mm².

### Example 1

### Co-Incubation of CD8⁺ Cells of Melanoma Patient with Labeled Autologous CD Cells Loaded with HR-gp100 or Melanoma Cells

Dendritic cells (DC) from a melanoma patient were loaded with a tumor protein (HR-gp100) and were used to sensitize autologous lymphocytes.

Briefly, CD8⁺ lymphocytes from a melanoma patient were separated from nonadherent MNC thawed the previous day, using magnetic beads (Miltenyi, Germany) according to the manufacturer's instructions. Autologous HR-gp100-loaded DC were incubated with CD8⁺ lymphocytes, for 48 hours at a 10:1 (lymphocytes:DC) ratio. After co-incubation, the lymphocytes and DC were washed and incubated with DiD and CMFDA-stained melanoma cells for further 4 hours. The cells were then collected, fixed, mounted on slides, and observed using a confocal laser scanning microscope (Zeiss, Model 410, Germany).

As demonstrated by the series of photographs shown in FIG. 1, the FITC-labeled HR-gp100 protein was being take-up by the loaded dendritic cells. FIG. 2 shows the interaction of a dendritic cell loaded with the HR-gp100 protein with CD8 lymphocytes obtained from melanoma patient. The subsequent FIG. 3, demonstrate clearly that during co-incubation of CD8⁺ lymphocytes from a melanoma patient and a DiD- and CMFDA-stained autologous melanoma cell, an unstained lymphocyte can be seen in the vicinity of a stained melanoma cell (FIG. 3A), and that attachment of a lymphocyte to a melanoma cell induced transfer of membranal stain from a melanoma cell to an autologous CD8⁺ lymphocyte (FIG. 4B).

Finally, FIG. 4 demonstrates clearly a DiD-labeled melanoma cell which shows signs of destruction after attachment to autologous CD8⁺ lymphocytes, previously activated with HR-gp100-loaded DC.

These results indicate that CD8⁺ obtained from melanoma patients rapidly captured membrane fragments from autologous DC which was loaded with HR-gp100. Therefore, isolation of these particular desired CTLs based on the presence of this antigen on their membrane, is feasible.

### Example 2

### Preparation and Sorting of MUC1 Specific CTLs

Tumor cells which express MUC1, were labeled with the lipophilic tracer DiD as indicated in Experimental procedures. The labeled cells were subsequently incubated with relevant lymphocytes for several hours, before sorting the DiD-labeled lymphocytes. Initially, in order to set up the system, a well-defined antigen-lymphocyte system available in the inventor's laboratory was used. A MUC1 peptide, D6 (LLLTVLTVV, as denoted by SEQ ID NO: 1) against which specific T cell clones have been produced, was loaded on T2 cells or on monocyte-derived dendritic cells obtained from an HLA-A2 donor. The antigen-loaded cells were then incubated in the presence of the T cell clone. This system was used for establishing optimal conditions for cell interaction and membrane transfer.

As will be indicated below, PBMC from patients, incubate with DiD-labeled autologous tumor cells, or with antigen-loaded dendritic cells were also used similarly.

### Example 3

### Interaction of Peripheral Blood Mononuclear Cells (PBMC) and Stimulating Cells

Enriched PBMC are obtained by cytopheresis from normal donors and from cancer patients. This procedure has been approved by Helsinki. PBMC are further purified on a Ficoll-Hypaque gradient, and frozen in working aliquots in liquid nitrogen.

For each experiment, an aliquot is thawed, and incubated with labeled stimulating cells. Co-incubation is performed at different cell ratios, as it has been shown that the antigen concentration used may affect the functional avidity of the cells [Bullock T N. et al., J. Immunol. 167, 5824-31 (2001)].

Initially, cells will be co-incubated for approximately 4 hours, but the length of time may vary depending upon experimental conditions and results obtained.

After co-incubation, the cell mixture is stained with CD8⁺-FITC, in a manner which does not affect the proliferating ability nor the biological activity of CD8⁺ cells. Double stained cells are then sorted under sterile conditions. The sorting procedure is repeated where the number of positive cells is below the sensitivity of the technique. The sorted cells are subsequently incubated in the presence of feeder cells, and propagated to large numbers in the presence of interleukin (IL)-2 and/or OKT3 antibody. The phenotype of the lymphocytes is determined by staining with specific monoclonal antibodies (CD3, CD4, CD8, CD56), followed by FACS analysis.

As will be shown in the following Examples, to determine biological activity of the cell population obtained, the following assays were performed:

(a) in vitro stimulation of lymphocytes with tumor targets and/or peptide loaded target cells (such as T2 cells, a HLA-A2.01 TAP-deficient lymphoblastoid line) for 24-48 hours, and determination of cytokine production (IFN-γ, IL-5, IL-10), by the stimulated lymphocytes.
(b) in vitro stimulation of lymphocytes with tumor targets and/or peptide loaded target cells for 5-7 days, and measurement of cytotoxic activity, using ³⁵S-methionine labeled target cells.

### Example 4

### Generation of Peptide-Reactive T Cell Lines

In order to further characterize the particular cell population having membrane-capture activity, T cell lines, specific for a particular peptide were next developed. T cell lines were generated from metastatic melanoma HLA-A*0201-positive patients that specifically recognized peptides from gp100 and Melan A/MART1. Briefly, gp100:209-217 (210M) (IMDQVPFSV, as denoted by SE ID NO:2), gp100:154-162 (KTWGQYWQV, as denoted by SE ID NO:3), and Melan-A/MART1: 27-35 (AAGIGILTV, as denoted by SE ID NO:4) were used for in vitro stimulation of donor lymphocytes in 96-well plates (microculture format). PBMCs were cultured (5×10⁵ cells/well) in complete medium ((CM), RPMI 1640, 2 mM L-glutamine, 50 units/ml penicillin, 50 μg/ml streptomycin (Invitrogen), and 10% heat-inactivated human AB serum) containing 10 μM peptide. Two days later, 300 IU/ml rIL-2 (Chiron Co.) were added and renewed every 3 days. On days 7 and 14, the cultures were re-stimulated with peptide-pulsed (10 μM peptide, 1×10⁶ cells/ml, 2-4 h incubation at 37° C.) autologous-irradiated (4000 rad) PBMCs. On day 21, each microculture was evaluated for specific peptide recognition on the basis of IFN-γ secretion in response to T2 cells (HLA-A*0201 peptide transporter-associated protein-deficient T-B hybrid) pre-incubated with peptide and 624mel cells (HLA-A*0201⁺ cell line that endogenously express gp100 and Melan A). Positive microcultures (>100 pg/ml and at least twice background with an irrelevant HLA-A*0201-restricted peptide) were re-stimulated individually in 24-well plates with 5×10⁶ peptide-pulsed autologous-irradiated PBMCs/well and further expanded using a rapid expansion protocol [Dudley M E. et al., Science 298, 850-4 (2002)].

Four specific T-cell lines were isolated and designated as follows: L-8 for Melan-A/MART1:27-35, L-9 for gp100:154-162, L-43 for gp100:209-217(210M) and L-33 for MUC1:63-71. These selected T cell lines were examined for IFN-γ secretion in response to stimulation with their specific peptides. As shown by FIG. 5A, the selected T cell lines secret IFN-γ in a peptide-specific manner following stimulation by correspondent peptide-loaded T2 cells and 624mel but not irrelevant (HIV-derived) peptide-loaded T2 cells and HLA-A2⁻ M181 melanoma cells. The generated cytotoxic T lymphocytes (CTLs) effectively lyse peptide-loaded T2 cells and, importantly, 624mel melanoma targets. The MUC1-derived, HLA-A*0201-restricted CTL epitope MUC1:63-71 was used to generate T cell line (L-33) representing melanoma-irrelevant effector cells.

### Example 5

### Intercellular Transfer of Membrane Fragments from Target Cells onto CD8⁺ T Lymphocytes

The absorption of professional antigen presenting cell determinants including MHC and co-stimulatory molecules on the effector cell surface has been reported (Huang et al, 1999). This process has been shown to occur as a very early event during the effector-target recognition in a T-cell receptor (TCR) dependent manner. To study whether CTLs may capture membrane fragments from artificial antigen presenting cells the specific peptide-reactive T cell lines prepared by the present invention were used as effectors and the corresponding and irrelevant peptide-loaded T2 cells as specific and non-specific targets, respectively. The transfer of a fluorescent lipophilic dye (1-1′-dioctodecyl-3,3,3′,3′-tetramethyl-indocarbocyanine perchlorate, DiI, Molecular Probes, Invitrogen) incorporated into the target cell membranes, to T cells was next analyzed. To this end, peptide-preloaded T2 cells were stained with DiI according to the manufacturer's instructions, washed five times and co-cultured in 24-well plates (2 ml complete medium/well) with 1-2×10⁶ effector cells at E:T ratio of 3:1. Following incubation for 1 to 5 h, cells were washed, stained with anti-CD8 or CD4 mAb (Ebioscience) and analyzed by flow cytometry. As shown in FIG. 6A, only CD8⁺ but not CD4⁺ lymphocytes from MelanA/Mart1:27-31-reactive T cell line, were able to capture membrane fragments from the specific peptide-loaded T2 cells. The transfer of the target membrane derivatives is peptide specific and therefore TCR-dependent, since there are no DiI-stained CD8 T cells following co-incubation with irrelevant HIV peptide-loaded T2 cells (FIG. 6A). The intercellular transfer of melanoma cell membrane fragments onto melanoma antigen peptide-specific CD8 T lymphocytes was next examined. HLA-A*0201⁺, gp100⁺ and MelanA/Mart1⁺ 624mel cells were stained and co-cultured with the specific T-cell lines of the invention, L-8, L-9 and L-43, as described above. The HLA-A*0201⁻ M181 melanoma cells served a negative control, whereas MUC1 peptide-reactive CD8⁺ lymphocytes (L-33 T-cell line) represent irrelevant effector cells. As clearly shown by FIG. 6B, melanoma antigen peptide-reactive CD8⁺ lymphocytes effectively capture membrane parts from the 624mel cells in HLA-restricted manner.

### Example 6

### DiI⁺CD⁺ T Lymphocytes Efficiently Kill Peptide-Loaded Target Cells Membrane Transfer as a Label of CD8 Lymphocyte Functional Cytotoxicity

The inventor's next goal was to examine whether membrane fragment scavenging by CD8 T lymphocytes has any functional implication in terms of actual cytotoxicity. Therefore, the cytotoxic activity of gp100:154-reactive DiI⁺CD8⁺ lymphocytes (labeled cells) was compared to that of DiI⁻ CD8⁺ cells (unlabeled cells) following co-incubation with DiI-stained 624mel melanoma cells. The DiI⁺ and the DiI⁻ CD8⁺ positive lymphocytes were sorted out by flow cytometry as shown by FIG. 7A. The sorted cells were expanded using a rapid expansion protocol and examined in CTL assay against specific (gp100:154) versus irrelevant (HIV) peptide-loaded T2 cells, as specific and non-specific targets, respectively. The MUC-1:63-reactive CD8⁺ lymphocytes served as melanoma-irrelevant effectors. As clearly demonstrated by FIG. 7B, only the double positive DiI⁺CD8⁺ lymphocyte population, that scavenged 624mel membrane fragments, constitute functional CTLs that destroyed their targets in a peptide-specific manner. The DiI⁻CD8⁺ lymphocytes were unable to lyse gp100:154-loaded T2 cells and demonstrated only background cytotoxicity.

### Example 7

### Selection and Expansion of Functional Cytotoxic T Cells out of Bulk PBMC

The inventors next attempted to select and expand cytotoxic lymphocyte populations directly from bulk PBMC of melanoma patients that received autologous melanoma cell vaccine. To enrich the tumor-reactive T-cell populations, PBMC were stimulated by irradiated (17000 rad) autologous melanoma cells in complete medium supplemented with 300 IU/ml IL-2. After 12 days, the bulk cultures were harvested and co-incubated with DiI-stained autologous melanoma cells at 37° C. for 6 h, stained by anti-CD8 mAb and analyzed by flow cytometry, as described above. As shown in FIG. 8A, a small but distinct population of CD8 lymphocytes (2.8% of total CD8⁺ cells) scavenges DiI-stained membrane fragments from the target cells. The 12-day in vitro stimulation by autologous melanoma cells was a mandatory step since no target membrane transfer has been evident for melanoma-unstimulated PBMC (FIG. 8A, left panel). DiI⁺CD8⁺ cells were sorted out and expanded in vitro by the rapid expansion protocol. The frequency of DiI⁺CD8⁺ cells increased from 2.3% to 23% of the expanded cells, a 10-fold enrichment (FIG. 8B). The expanded cells were next analyzed for cytotoxic activity against M199 autologous melanoma cells. K562 cells were used as control excluding natural killer-like activity and autologous CD4⁺ cells were used as autologous non-melanoma targets. As clearly revealed by FIG. 8C, the expanded cells originating from the DiI⁺ population showed significant cytotoxic activity against specific targets but not against non-specific targets.

### Example 8

### Correlation Between Membrane Exchange and CD107A Expression

In order to further characterize CD8⁺DiI⁺ lymphocytes, lymphocytes that underwent membrane exchange were stained for CD107A (lysosomal-associated membrane protein 1), which is known to be expressed as a result of degranulation of lymphocytes.

The results demonstrated by FIG. 9, show that almost all CD8+DiI^{high} cells (subgate 1) are highly cytolytic and melanoma specific lymphocytes. More than 92% of them immobilized CD107A on their cell surface following 1 hour co-incubation with autologous melanoma.

The CD8⁺DiI^{low} population consists of mostly anergized lymphocytes (>70% of them are CD107A negative). Recently, the inventors discovered that DiI⁺ and DiI⁻ CD8 lymphocytes are of the same TCR, by experiments with tetramer staining of Melan A-reactive lymphocyte line. Therefore, without being bound by any theory, the inventors may assume that DiI⁻ CD8⁺ cells represent not only tumor-irrelevant lymphocytes, but also functionally impaired tumor-specific cells.

In conclusion, the present invention clearly demonstrates that CD8⁺ lymphocytes scavenging target membrane fragments are functional cytotoxic cells. These cells can be sorted out of bulk lymphocyte culture and be further expanded in vitro. The expanded cells preserve their cytotoxic capacity and are therefore of potential use for adoptive cell therapy.

### Example 9

### Establishment of Human Melanoma Therapeutic Model in Nude Mice

The results of the invention presented by Examples 1 to 8, encouraged the inventors to examine the feasibility of using the isolated antigen-specific cytotoxic T lymphocytes of the invention in adoptive cell therapy. Therefore, the inventors have next established a human melanoma (624mel) model in nude mice, as described in Experimental procedures. Based on its ability to form homogeneous tumors following s.c challenge of 10⁶ cells, the 624mel/nude mouse model is used for adoptive cell therapy studies for sorted and expended melanoma-specific DiI⁺CD8⁺ double positive lymphocytes. Briefly, the melanoma-specific DiI⁺CD8⁺ double positive lymphocytes cells are sorted out of bulk lymphocyte culture following co-incubation with DiI-stained 624mel melanoma cells and further expansion in vitro. As shown herein before, DiI⁻CD8⁺ lymphocytes represent functionally impaired effector cell population and therefore are served as control. Unsorted CD8⁺ lymphocytes are expanded and used as an additional control. The expanded cells are transferred into human 624mel melanoma-bearing immuno-compromised CD1^{nude/nude} mice as follows: seven days after tumor induction, 5×10⁶ CD8⁺ T cells are injected around the tumors. The adoptive transfer is followed by local s.c. administration of 1000 U rhIL-2 twice a day for 5 days. The effect of DiI⁺CD8⁺ lymphocytes on tumor growth is compared to that of DiI⁻ and unsorted CD8⁺ lymphocytes by monitoring tumor sizes and mouse survival.

1-23. (canceled)

24. A method for the preparation and isolation of an immune system cell which is capable of specifically recognizing an antigen related to a pathologic disorder, which method comprises the steps of:
(a) providing target cells expressing or presenting an antigen related to said pathologic disorder, or any fragment or peptide thereof;
(b) labeling said target cells with a suitable membrane detectable label;
(c) providing a sample comprising immune system cells;
(d) co-incubating the labeled target cells obtained in step (b) with the cells provided in step (c) for a suitable period of time under suitable conditions;
(e) identifying a population of immune system cells expressing the CD8⁺ antigen, from the co-incubated cells obtained in step (d), by a suitable means;
(f) selecting from the CD8⁺ antigen expressing cells identified in step (e) cells stained with said membrane-label using suitable means under suitable conditions, wherein said selected cells are CD8⁺/membrane-label double positive cells;
(g) propagating the CD8⁺/membrane-label double positive cells selected in step (f), under suitable conditions; and
(h) evaluating the biological activity of the cells obtained in step (g) on an end-point indication using a suitable test system, whereby difference in said end point compares to a suitable control is indicative of the biological activity of said cells.

25. The method according to claim 24, wherein said immune system cell is a lymphocyte, preferably, CTL (Cytotoxic T Lymphocyte).

26. The method according to claim 25, wherein said sample comprising immune system cell is a sample of autologous PBMC (peripheral blood mononuclear cells) obtained from a subject suffering from said pathologic disorder.

27. The method according to claim 24, wherein said target cells are cells obtained from a subject suffering from said pathologic disorder.

28. The method according to claims 24, wherein said pathologic disorder is an immune-related disorder selected from any one of a malignant disorder, pathologic viral or bacterial infection, an inflammatory disorder and an autoimmune disorder.

29. The method according to claim 28, wherein said immune related disorder is a malignant disorder and said target cell is a tumor cell obtained from any one of a primary tumor and a cell line.

30. The method according to claim 29, wherein said malignant disorder is selected from melanoma, carcinoma, leukemia, sarcoma and lymphoma.

31. The method according to claim 24, wherein said suitable means for identifying CD8⁺ expressing cells according to step (e) is addition of an antibody specific for CD8.

32. The method according to claim 24, wherein said target cell is an antigen presenting cell (APC) expressing or presenting said specific antigen, or fragments or peptides thereof.

33. The method according to claim 32, wherein said APC is loaded or transfected with an antigen specific for said pathologic disorder, which antigen is provided as any one of a peptide, a purified recombinant protein, a fusion protein, a nucleic acid construct encoding for said antigen, a cell lysate, supernatant or any preparation of a host cell expressing said construct, a cell line and tissue endogenously expressing said antigen.

34. The method according to claim 33, wherein said malignant disorder is melanoma.

35. The method according to claim 24, wherein said suitable test system for evaluating the biologic activity of propagated selected cells according to step (h), is any one of in vitro/ex vivo cell culture and in vivo animal model.

36. The method according to claim 35, wherein said test system is in vitro/ex-vivo cell culture of said antigen specific immune system cell and said end point indication is any one of cytokine production and cytotoxic activity of said cells in response to stimulation with said target cells.

37. The method according to claim 35, wherein said test system is in-vivo animal model of CD1^{nu/nu} nude mice and said end point indication is inhibition of tumor growth and increase in mice survival by said cells as compared to a suitable control.

38. A cytotoxic T cell lymphocyte (CTL) specific for an antigen related to a pathologic disorder, wherein said CTL is isolated by the method according to claim 24.

39. A pharmaceutical composition for the treatment of a pathologic disorder comprising as an active ingredient a lymphocyte capable of recognizing an antigen specific for said pathologic disorder, said composition optionally further comprising at least one pharmaceutically acceptable additive, carrier, excipient and stabilizer, and other therapeutic constituent/s.

40. The composition according to claim 39, wherein said lymphocyte is CTL (Cytotoxic T Lymphocyte).

41. A composition for the treatment of a pathologic disorder comprising as an active ingredient a lymphocyte capable of recognizing an antigen specific for said pathologic disorder, said composition optionally further comprising at least one pharmaceutically acceptable additive, carrier, excipient and stabilizer, and other therapeutic constituent/s, wherein said lymphocyte is obtained by the method defined in claim 24.

42. The composition according to claim 41, wherein said pathologic disorder is an immune related disorder selected from any one of a malignant disorder, pathologic viral or bacterial infection, an inflammatory disorder and an autoimmune disorder.

43. The composition according to claim 42, wherein said immune-related disorder is a malignant disorder selected from melanoma, carcinoma, leukemia, sarcoma and lymphoma, preferably, melanoma.

44. A method of treatment a pathologic disorder in a subject in need thereof, comprising the step of administering to said subject a therapeutically effective amount of a lymphocyte capable of specifically recognizing an antigen related to said pathologic disorder or of a composition comprising the same.

45. A method of treatment a pathologic disorder in a subject in need thereof, comprising the step of administering to said subject a therapeutically effective amount of a lymphocyte capable of specifically recognizing an antigen related to said pathologic disorder or of a composition comprising the same, wherein said lymphocyte is obtained by the method defined in claim 24.

46. A method for preparing a therapeutic composition for the treatment of a pathologic disorder in a subject in need thereof, which method comprises the steps of:
a) isolating a cytotoxic T cell lymphocyte specific for an antigen related to said pathologic disorder, by the method according to claim 24;
b) admixing said lymphocyte with at least one of a pharmaceutical acceptable carrier, diluent, excipient and/or additive.
